(19) United States
(12) Patent Application Publication
(10) Pub. No.: US 2024/0293460 A1
(43) Pub. Date: Sep. 5, 2024

(54) DKK1/HLA-A2 BINDING MOLECULES AND METHODS OF THEIR USE

(71) Applicant: THE METHODIST HOSPITAL SYSTEM, Houston, TX (US)

(72) Inventors: Qing YI, Houston, TX (US); Jianfei QIAN, Pearland, TX (US)

(21) Appl. No.: 18/566,129

(22) PCT Filed: Jun. 2, 2022
(86) PCT No.: PCT/US2022/031926

§ 371 Date: Dec. 1, 2023

Related U.S. Application Data
(60) Provisional application No. 63/195,954, filed on Jun. 2, 2021.

(51) Int. Cl. A61K 35/17; A61K 39/00; A61P 35/00; C07K 16/18; G01N 33/574

(52) U.S. Cl. CPC A61K 35/17; A61K 39/4611; A61K 39/4631; A61K 39/464402; A61P 35/00; C07K 16/18; G01N 33/57492; C07K 2317/24; C07K 2317/56; G01N 2333/705

(57) ABSTRACT

Disclosed are antigen binding molecules that bind to Dickkopf-1 (DKK1) P20 peptide in the context of MHC-HLA-A2 (i.e., a DKK1-A2 complex). Such antigen binding molecules can by antibodies, antibody fragments, bi-specific antibodies, immunotoxins or the chimeric antigen receptor portion of a chimeric antigen receptor T cell. Also disclosed herein are methods of using said antigen binding molecules for the treatment of cancer.

### I. CROSS-REFERENCE TO RELATED APPLICATIONS

This application claims the benefit of U.S. Provisional Application No. 63/195,954, filed on Jun. 2, 2021, which is incorporated herein by reference in its entirety.

### II. BACKGROUND

Pancreatic cancer, i.e., pancreatic ductal adenocarcinoma (PDAC), is one of the most lethal human malignancies ranking 4^{th} among cancer-related death in the United States. Potent therapeutic options are lacking. Immunotherapies using the immune checkpoint blockade (ICB) have transformed the outlook of advanced patients with melanoma, lung cancer and others, PDAC is, however, almost entirely refractory to FDA-approved ICB.

Similarly, lung cancer is the second most common cancer and the leading cause of cancer death for men and women, and non-small-cell lung cancer (NSCLC) accounts for 84% of lung cancers1. Progress in the treatment of advanced NSCLC over the last decade has been modest, and the 5-year overall survival rate remains low (25%) in the US. Although immunotherapy using the immune checkpoint inhibitors (ICB) against PD-1 or PD-L1 has moved from an intriguing salvage therapy to part of standard first-line care for advanced NSCLC patients, the response rates for PD-1 or PD-L1 monotherapy are low (≈20%). Moreover, many of the initial responders will eventually develop resistance to the therapy.

Although chimeric antigen receptor (CAR)-T cell therapy is gaining traction as one of the most promising advances in cancer immunotherapy, current CAR-T cell clinical trials in PDAC failed to improve survival and were unable to demonstrate any significant response. The failure of CAR T cell therapy was also observed in NSCLC, and at present, no effective CAR-T cell therapy has been developed for NSCLC. The unique challenges posed to CAR-T cell therapy in solid tumors include, but are not limited to, finding good tumor antigen(s), CAR-T cell infiltrating into and surviving in the tumor microenvironment (TME), and reducing toxicities. Overcoming these challenges is thus essential for the development of clinically efficacious CAR-T cell therapy for solid tumor patients. What are needed are new immunotherapies that can successfully target and treat pancreatic cancer.

### III. SUMMARY

Disclosed are methods and compositions related to antigen binding molecules that specifically bind to Dickkpf-1 (DKK1) peptide P20 in the context of HLA-A2.

In one aspect disclosed herein are antigen binding molecules (such as, for example, RNAi, peptide, protein, chimeric antigen receptor (CAR) T cell, CAR NK cell, CAR macrophage (CARMA), siRNA, immunotoxin, diabody, antibody, or a functional antibody fragment) that selectively binds to Dickkpf-1 (DKK1) peptide P20 in the context of HLA-A2.

Also disclosed herein are antigen binding molecules of any preceding aspect, comprising one or more of the heavy chain complementarity determining regions as set forth in SEQ ID NO: 1, SEQ ID NO: 2, SEQ ID NO: 3, SEQ ID NO: 7, SEQ ID NO: 8 SEQ ID NO: 9, SEQ ID NO: 25. SEQ ID NO: 26, and/or SEQ ID NO: 27 (such as, for example, antigen binding molecules comprising a heavy chain complementarity determining regions (CDRs) 1, 2, and 3 as set forth in SEQ ID NO: 1, SEQ ID NO: 2, SEQ ID NO: 3, respectively, including, but not limited to a heavy chain variable domain as set forth in SEQ ID NO: 14; antigen binding molecules comprising a heavy chain complementarity determining regions (CDRs) 1, 2, and 3 as set forth in SEQ ID NO: 7, SEQ ID NO: 8, SEQ ID NO: 9, respectively, including, but not limited to a heavy chain variable domain as set forth in SEQ ID NO: 18; or antigen binding molecules comprising a heavy chain complementarity determining regions (CDRs) 1, 2, and 3 as set forth in SEQ ID NO: 25, SEQ ID NO: 26, SEQ ID NO: 27, respectively, including, but not limited to a heavy chain variable domain as set forth in SEQ ID NO: 22) and/or one or more of the light chain complementarity determining regions as set forth in SEQ ID NO: 4, SEQ ID NO: 5, SEQ ID NO: 6, SEQ ID NO: 10, SEQ ID NO: 11, SEQ ID NO: 12, SEQ ID NO: 28, SEQ ID NO: 29, and/or SEQ ID NO: 30 (such as, for example, antigen binding molecules comprising a light chain complementarity determining regions (CDRs) 1, 2, and 3 as set forth in SEQ ID NO: 4, SEQ ID NO: 5, and SEQ ID NO: 6, respectively, including, but not limited to antigen binding molecules comprising a light chain variable domain as set forth in SEQ ID NO: 16; antigen binding molecules comprising a light chain complementarity determining regions (CDRs) 1, 2, and 3 as set forth in SEQ ID NO: 10, SEQ ID NO: 11, and SEQ ID NO: 12, respectively, including, but not limited to antigen binding molecules comprising a light chain variable domain as set forth in SEQ ID NO: 20; or antigen binding molecules comprising a light chain complementarity determining regions (CDRs) 1, 2, and 3 as set forth in SEQ ID NO: 428, SEQ ID NO: 29, and SEQ ID NO: 30, respectively, including, but not limited to antigen binding molecules comprising a light chain variable domain as set forth in SEQ ID NO: 24). For example, the antigen binding molecule can comprise SEQ ID NO: 14 and SEQ ID NO: 16; SEQ ID NO: 18 and SEQ ID NO: 20; or a humanized variable heavy chain and humanized variable light chain as set forth in SEQ ID NO: 22 and SEQ ID NO: 24. In some aspects, the antigen binding molecule can be conjugated to a detectable label (such as, for example, a fluorescent dye, a member of a binding pair, a metal, or an epitope tag).

In one aspect disclosed herein are antigen binding molecules of any preceding aspect, wherein antigen binding molecule comprises a CAR T cell that comprises a Th9 polarized T cell.

Also disclosed herein are antigen binding molecules of any preceding aspect, wherein antigen binding molecule comprises a bi-specific antibody (including, but not limited to a BiTE, Cross BiTE, and/or Tand BiTE) wherein the first antigen binding molecules bind DKK1 peptide P20 in the context of MHC HLA-A2 and the second antigen binding molecule binds CD3.

In one aspect, disclosed herein are hybridoma cell lines expressing the antigen binding molecule of any preceding aspect.

Also disclosed herein are methods of treating, inhibiting, reducing, decreasing, ameliorating, and or preventing a cancer and/or metastasis (such as, for example, pancreatic cancer, non-small cell lung carcinoma, breast cancer, myeloma, or leukemia) in a subject comprising administering to the subject any of the antigen binding molecules of any preceding aspect. For example, disclosed herein are methods of treating, inhibiting, reducing, decreasing, ameliorating, and or preventing a cancer and/or metastasis (such as, for example, pancreatic cancer, non-small cell lung carcinoma, breast cancer, myeloma, or leukemia) in a subject comprising administering to the subject a therapeutically effective amount of an antibody, bi-specific antibody, immunotoxin, or CAR T cell that specifically binds to DKK1 peptide P20 in the context of MHC HLA-A2.

In one aspect, disclosed herein are methods of detecting a cancer in a subject comprising a) obtaining a tissue sample of a suspected cancerous tissue from the subject; and b) assaying (such as, for example, by enzyme linked immunosorbent assays (ELISAs), enzyme linked immunospot assay (ELiSpot). radioimmunoassays (RIA), radioimmune precipitation assays (RIPA), immunobead capture assays, and/or flow cytometry) for the presence or overexpression of DKK1 with any of the antigen binding molecules of any of claims 1-6; wherein the presence of the DKK1 in the tissue sample not present in a negative control tissue sample or overexpression of the DKK1 in the tissue sample relative to a negative control tissue sample indicates the presence of a cancer in the subject.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show the DKK1 expression profile in normal tissues. FIG. 1A shows mRNA expression of DKK1 in normal tissues according to HPA RNA-seq normal tissues data (BioProject: PRJEB4337). Reads Per Kilobase of transcript, per Million mapped reads (RPKM) is a normalized unit of transcript expression. FIG. 1B shows the DKK1 gene expression in normal tissues by RT-PCR. FIG. 1C shows immunohistochemistry (IHC) staining with DKK1 monoclonal antibody (mAb) in pancreas tissue from ten healthy donors.

FIGS. 2A, 2B, and 2C show the DKK1 expression profile in cancer tissues. FIG. 2A shows a comparison DKK1 (mRNA) expression profile between tumor sample and normal tissue from TCGA datasets. FIG. 2B shows DKK1 gene expression in solid tumors (Oncomine data). FIG. 2C shows DKK1 gene expression in hematological malignancies (Oncomine data).

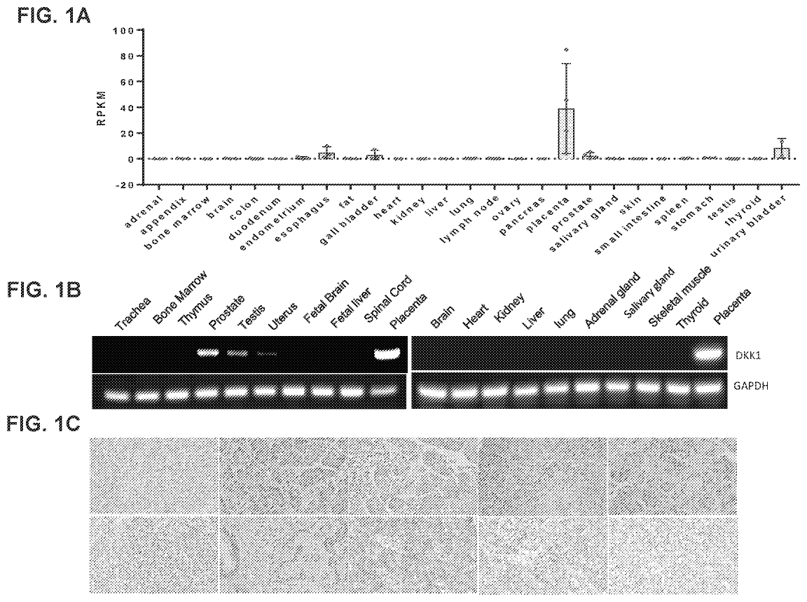

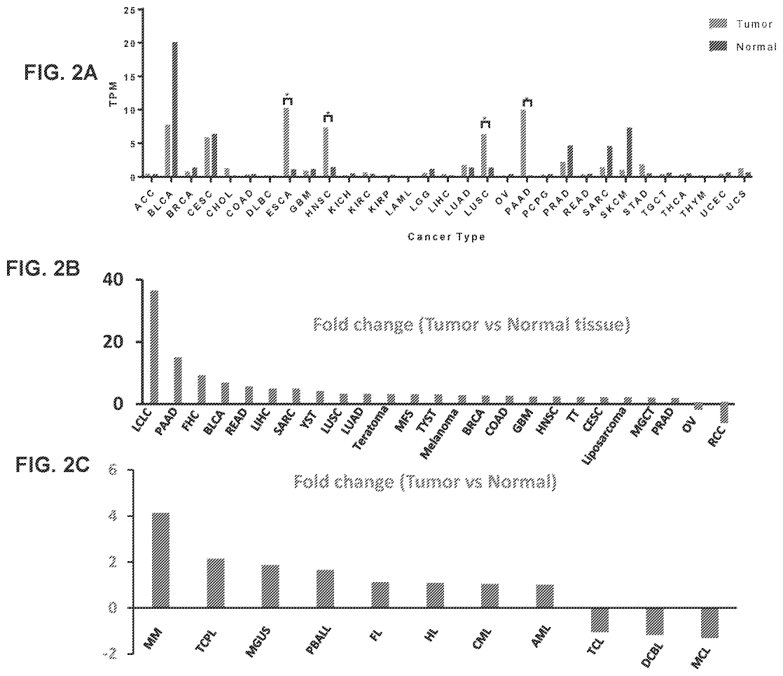

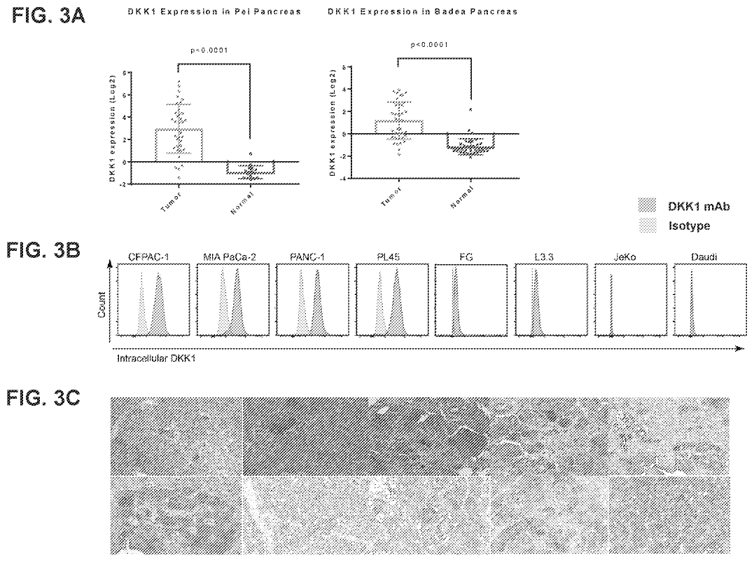

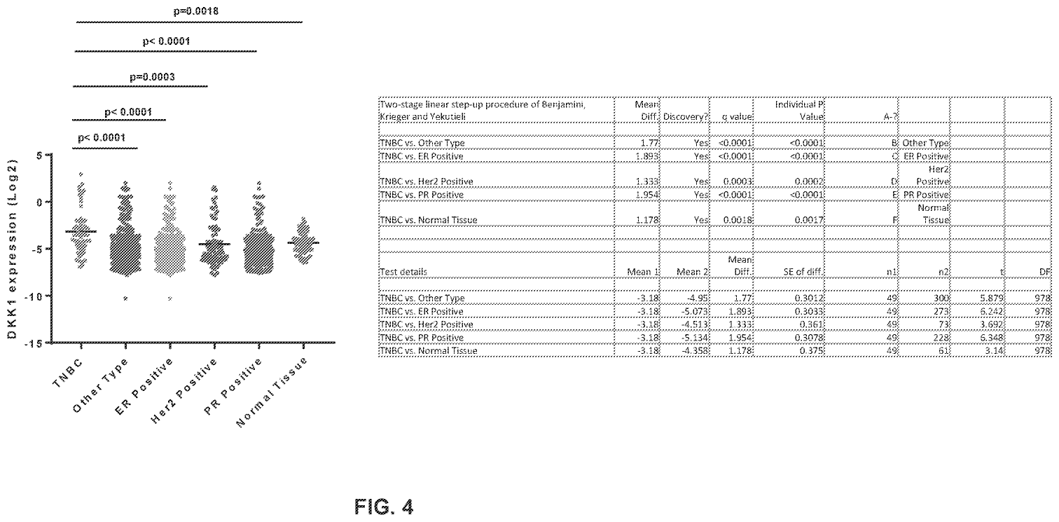

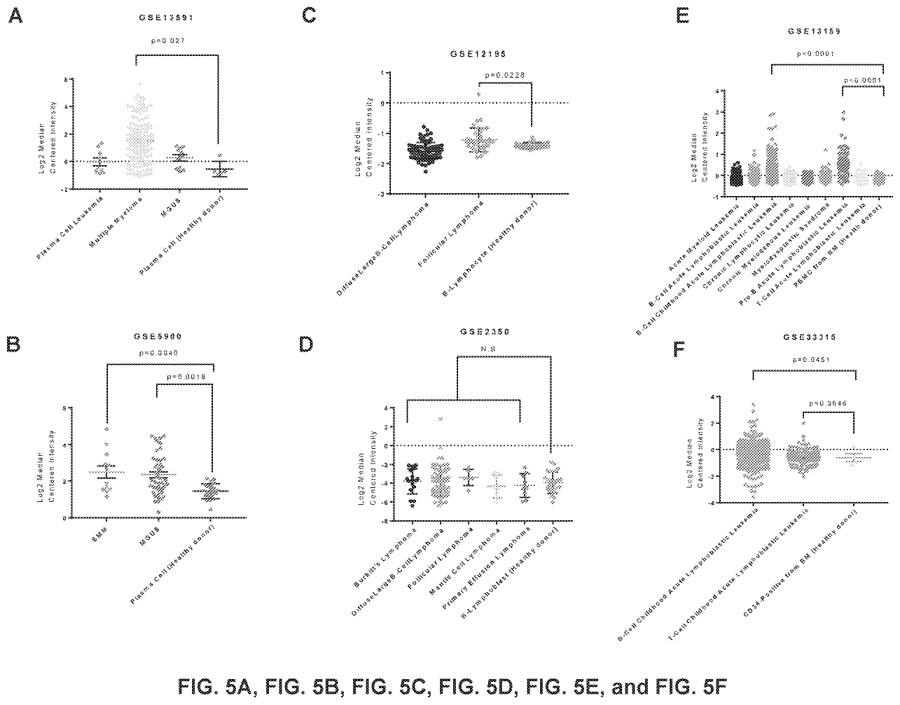

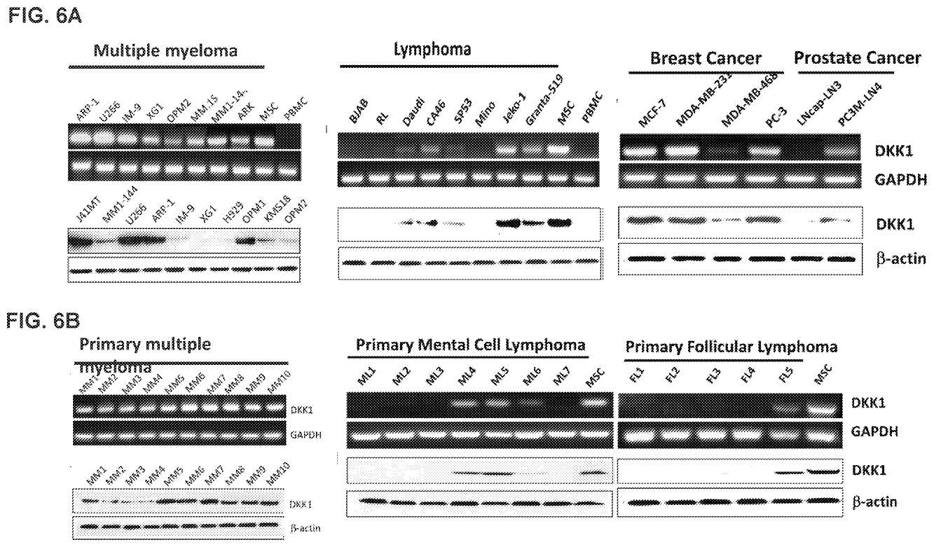

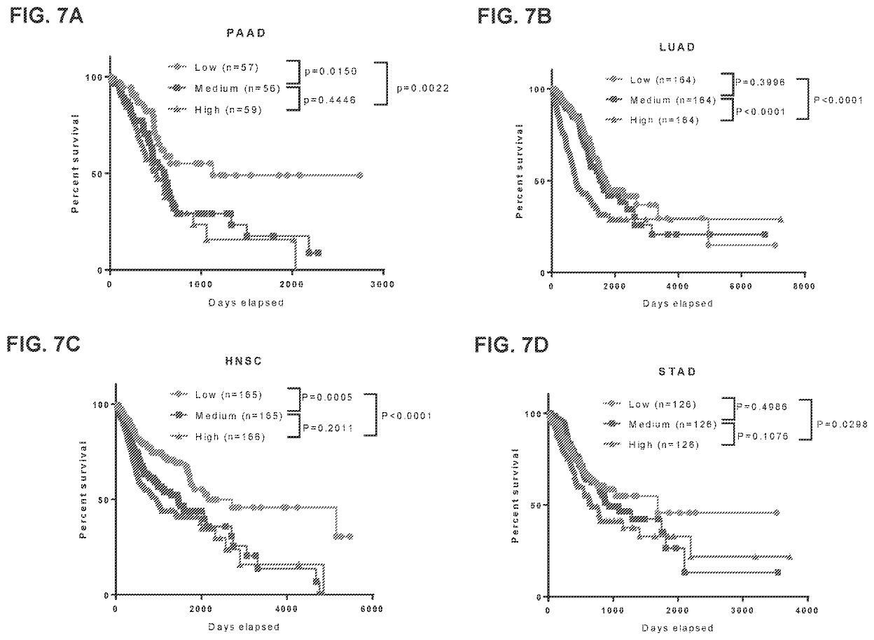

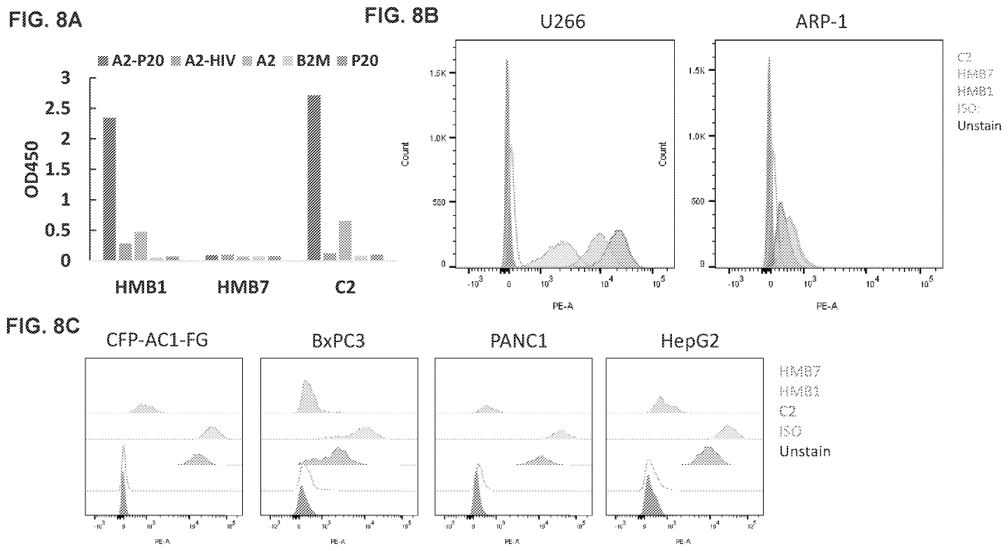

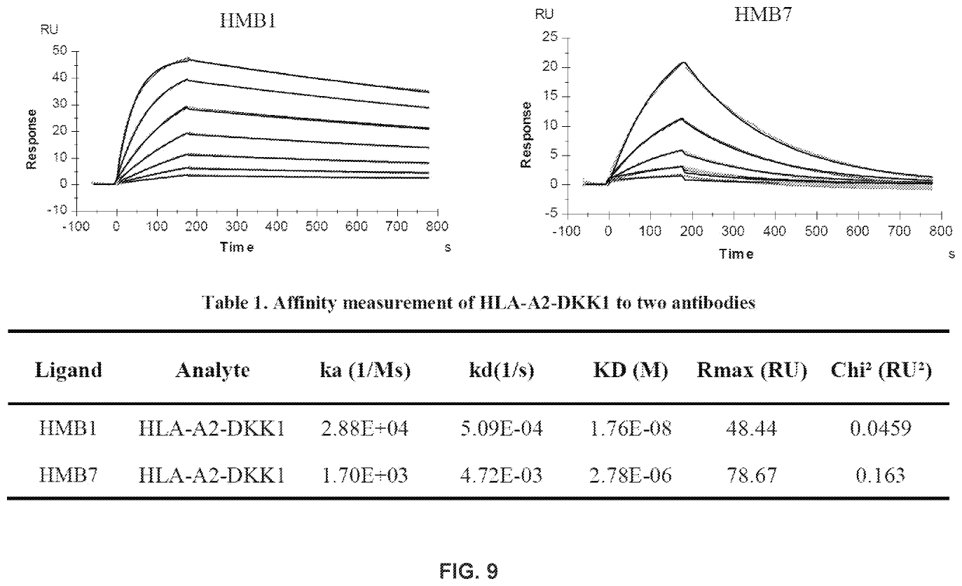

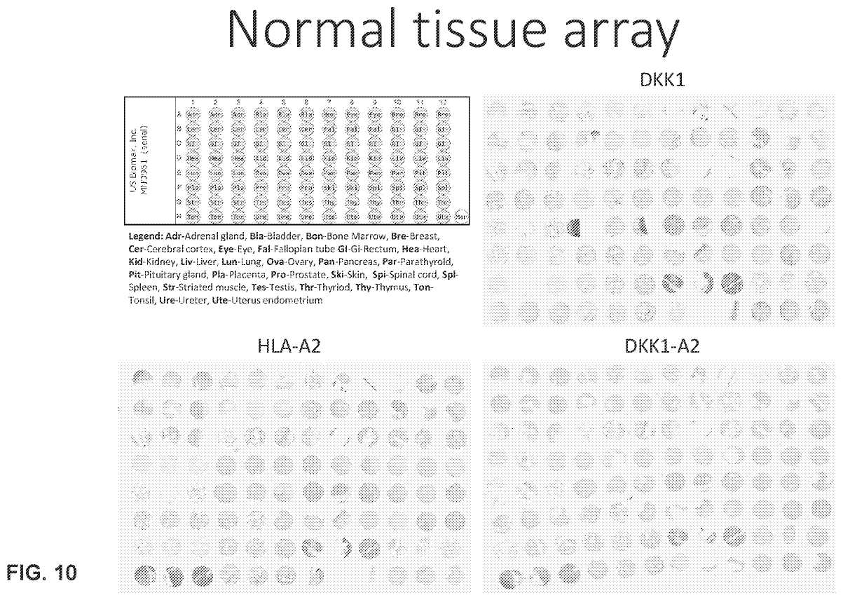

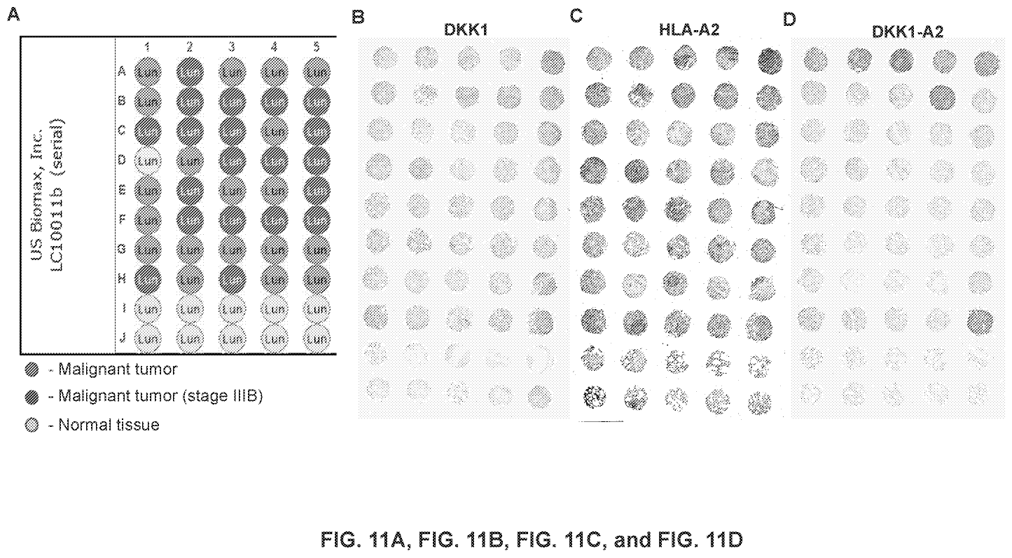

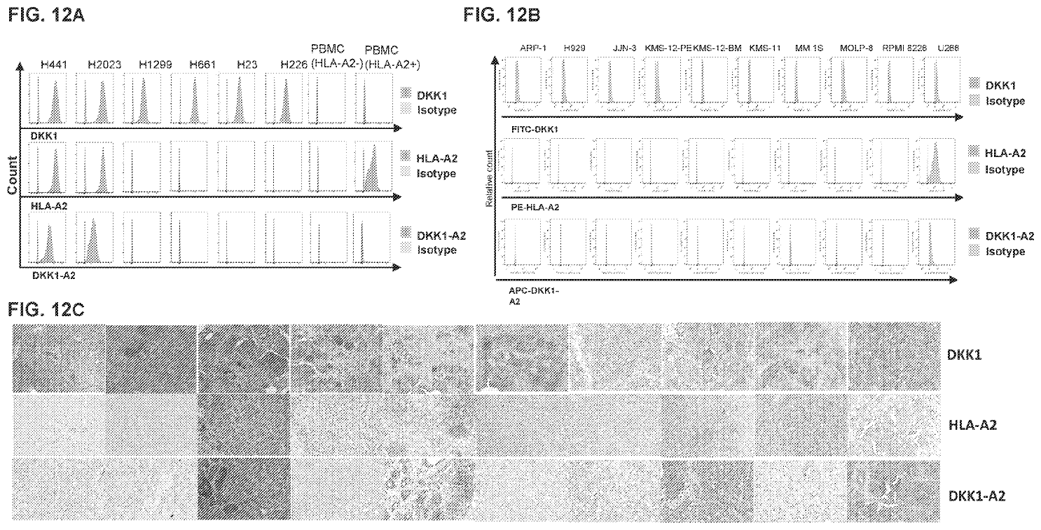

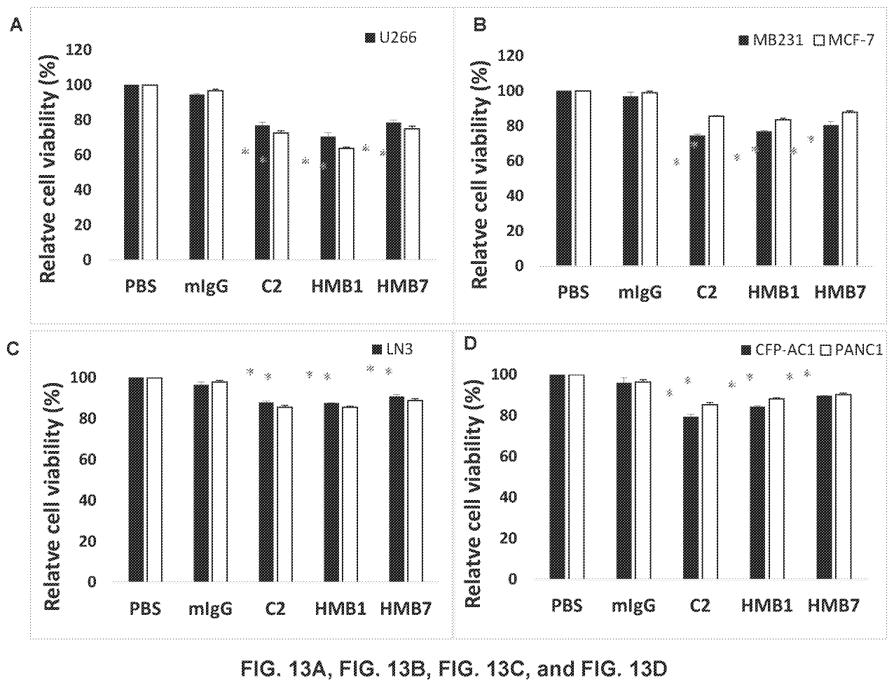

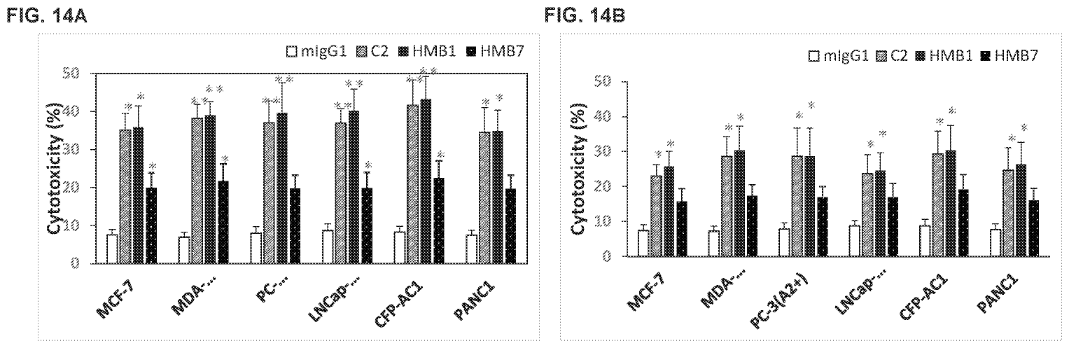

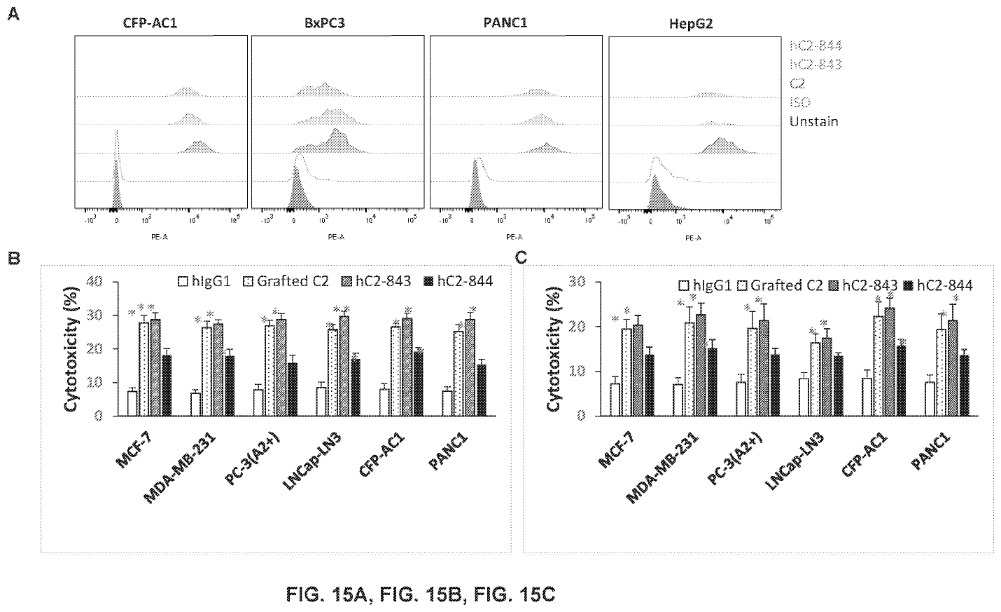

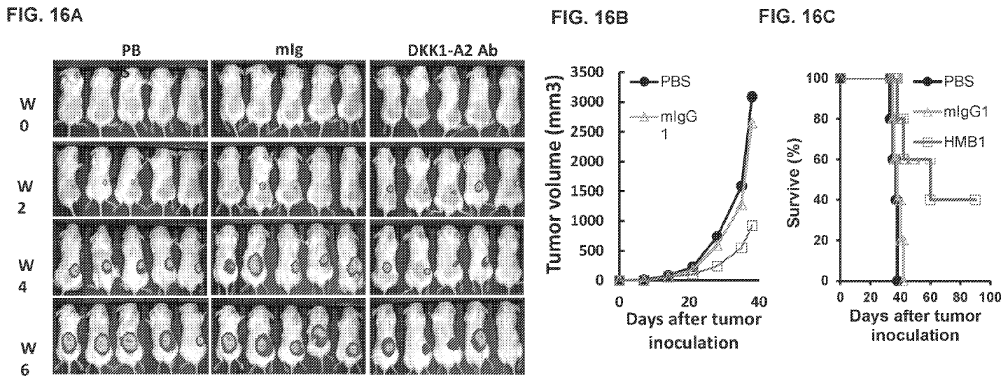

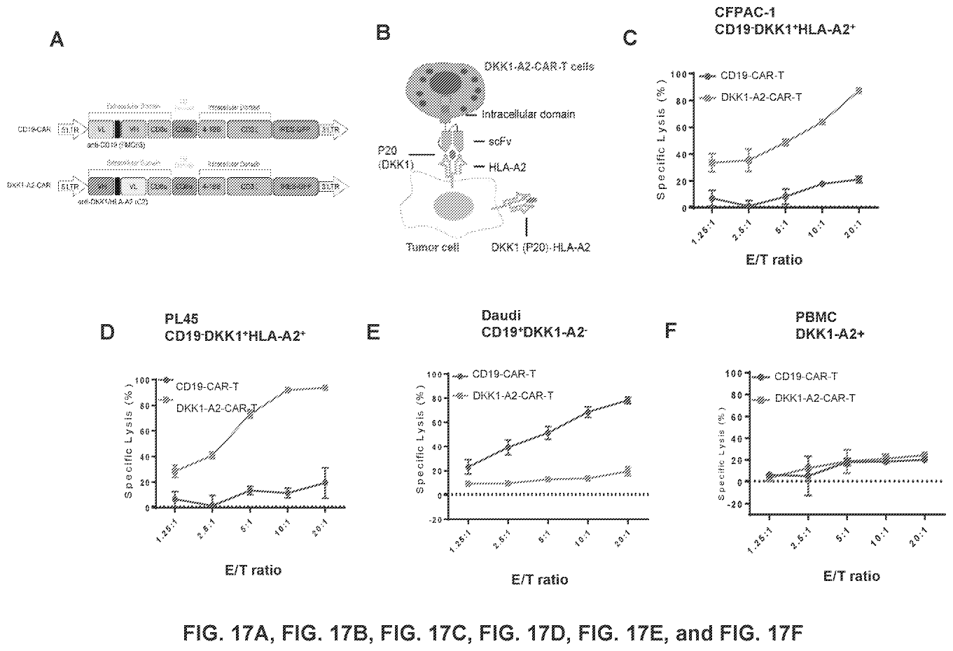

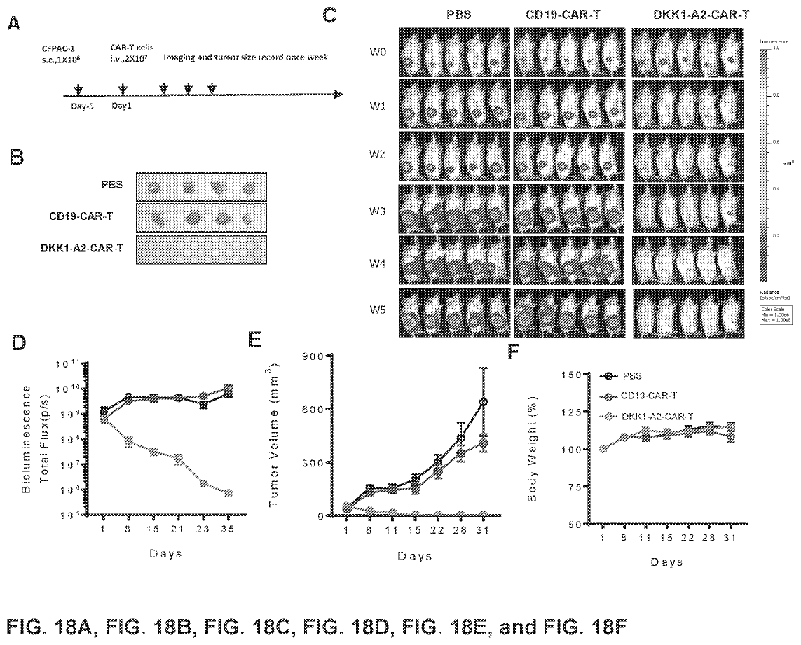

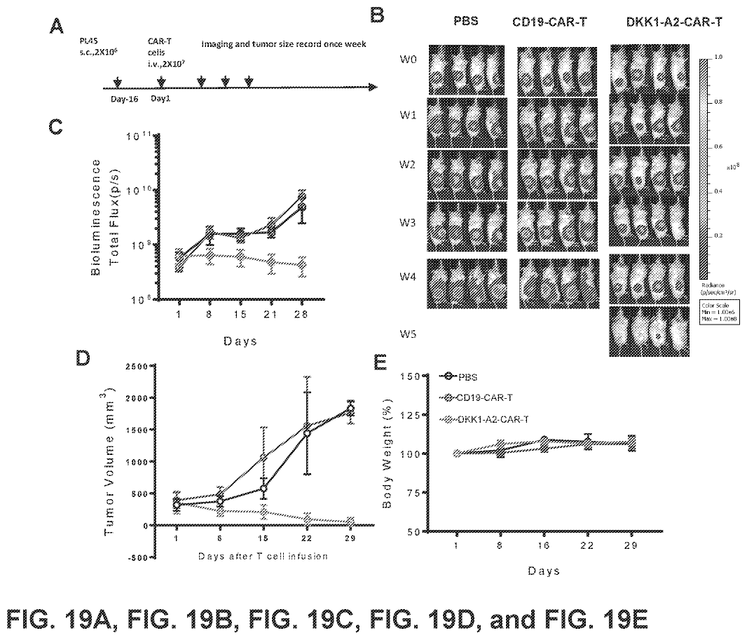

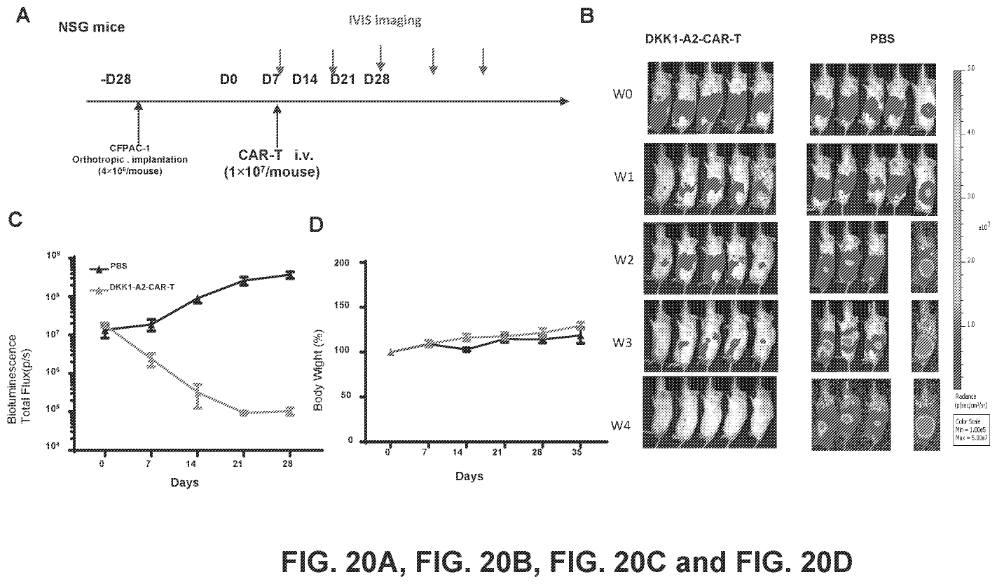

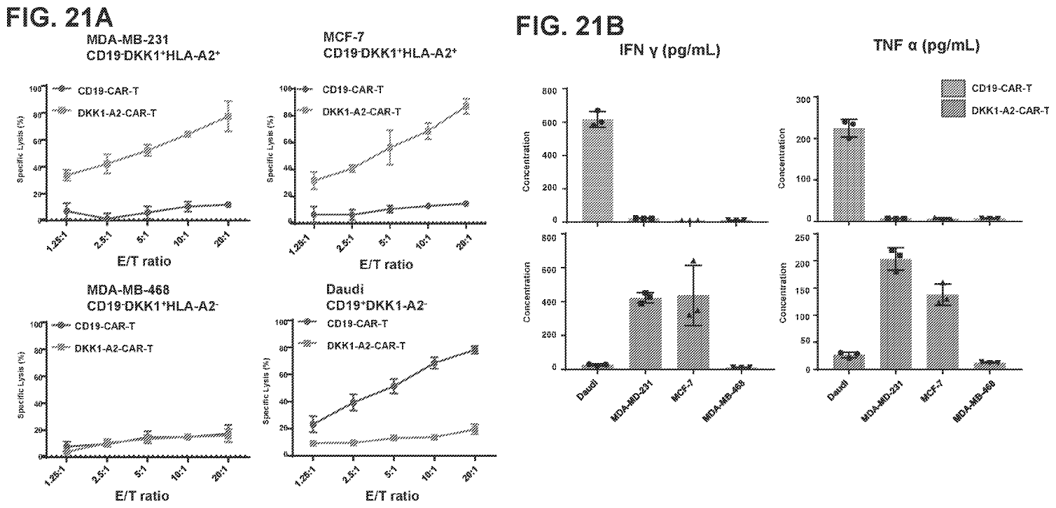

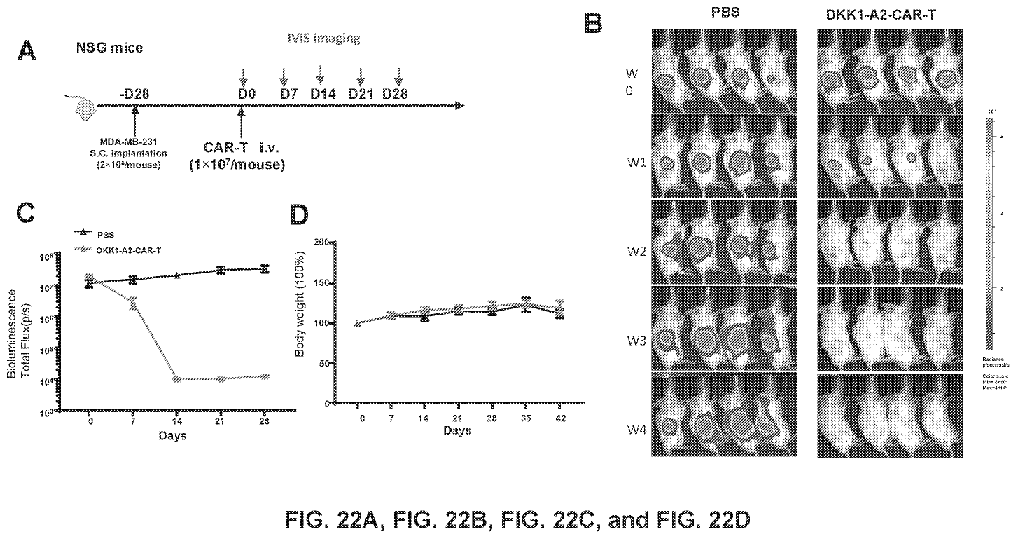

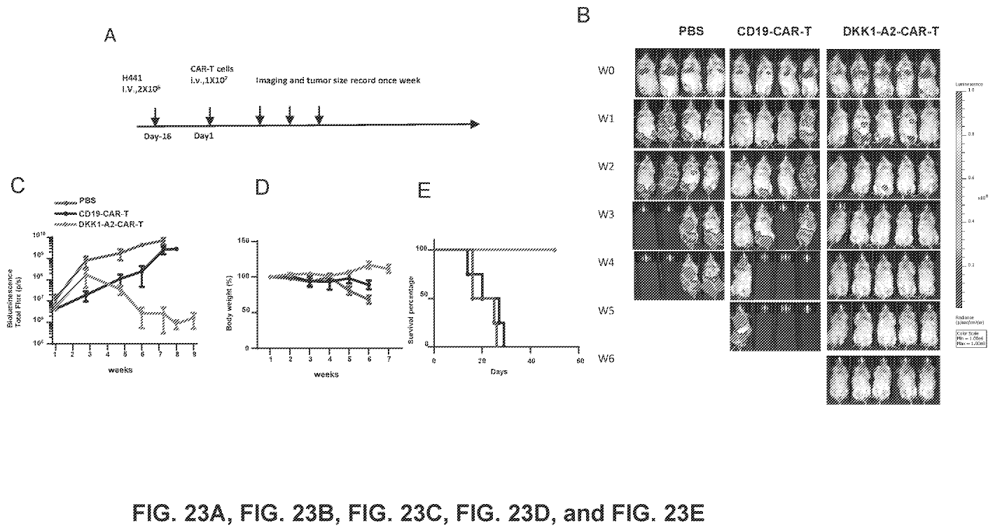

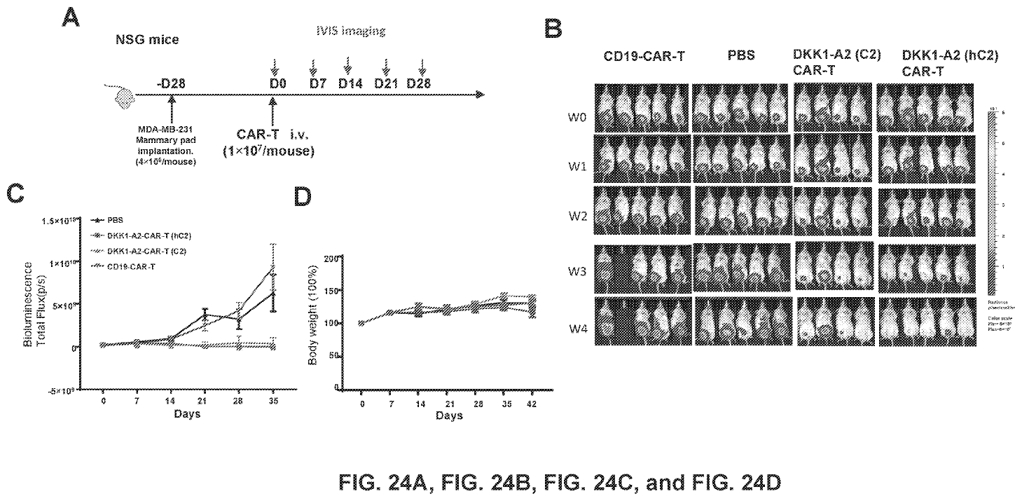

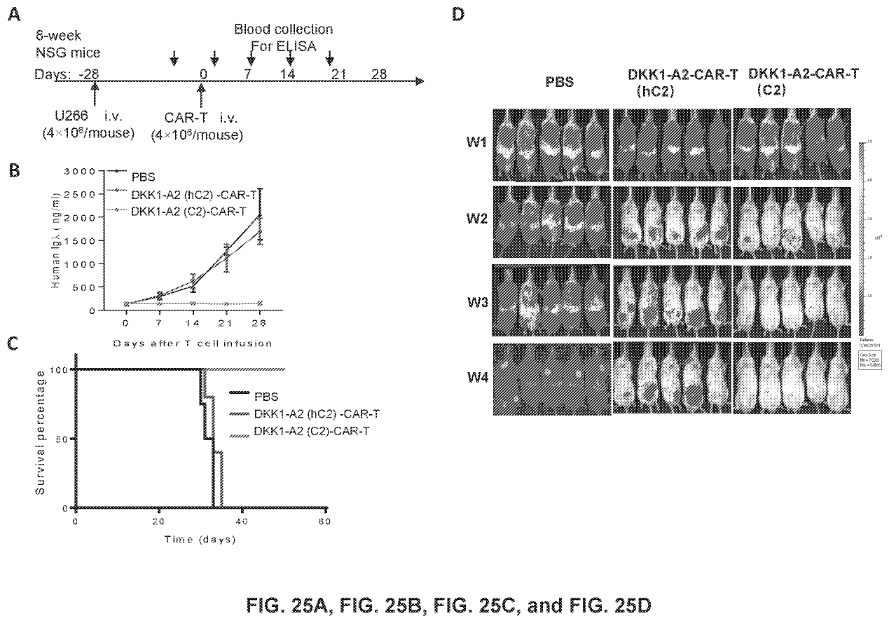

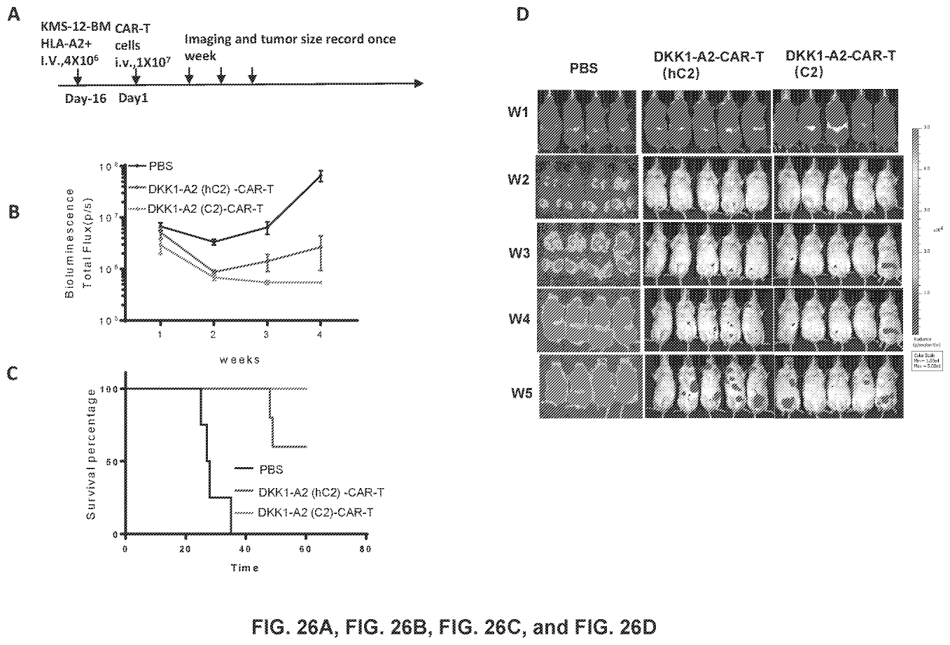

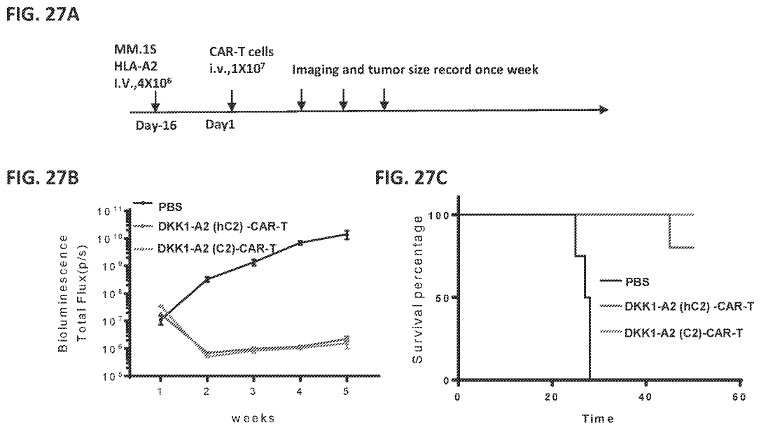

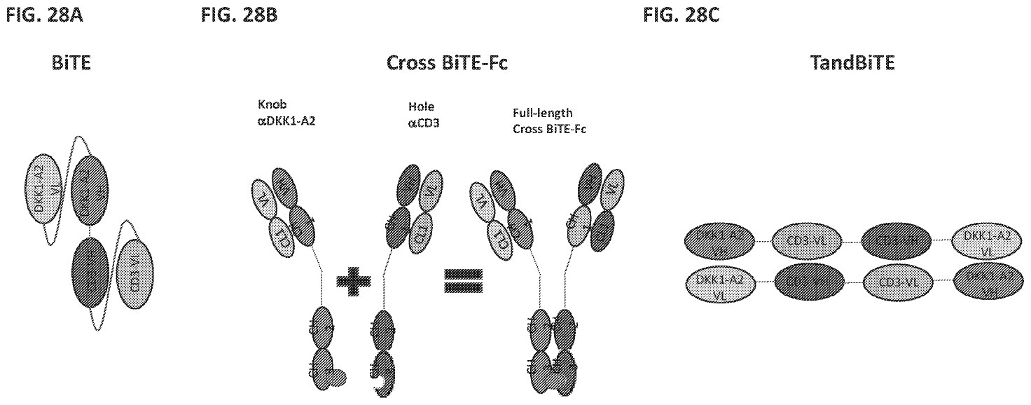

Abbreviations: ACC: Adrenocortical carcinoma, AML: Acute Myeloid Leukemia, BLCA: Bladder Urothelial Carcinoma, BRCA: Breast invasive carcinoma, CESC: Cervical squamous cell carcinoma and endocervical adenocarcinoma, CHOL: Cholangiocarcinoma, CML: Chronic Myelomonocyte Leukemia, COAD: Colon adenocarcinoma, DLBC: Lymphoid Neoplasm Diffuse Large B-cell Lymphoma, ESCA: Esophageal carcinoma, FHC: Fibrous Histiocytoma, FL: Follicular Lymphoma, GBM: Glioblastoma multiforme, HL: Hodgkin's lymphoma HNSC: Head and Neck squamous cell carcinoma, KICH: Kidney Chromophobe, KIRC: Kidney renal clear cell carcinoma, KIRP: Kidney renal papillary cell carcinoma, LAML: Acute Myeloid Leukemia, LGG: Brain Lower Grade Glioma, LIHC: Liver hepatocellular carcinoma, LCLC: Large Cell Lung Carcinoma, LUAD: Lung adenocarcinoma, LUSC: Lung squamous cell carcinoma, MCL: Mantle Cell lymphoma, MFS: Myxofibrosarcoma, MGCT: Mixed Germ Cell Tumor, MGUS: Monoclonal Gammopathy of Undertermined Significance, MM: Multiple myeloma, OV: Ovarian serous cystadenocarcinoma, PAAD: Pancreatic adenocarcinoma, PBALL: Pro-B Acute Lymphoblastic Leukemia, PCPG: Pheochromocytoma and Paraganglioma, PRAD: Prostate adenocarcinoma, READ: Rectum adenocarcinoma, RCC: Renal Cell Carcinoma, SARC: Sarcoma, SKCM: Skin Cutaneous Melanoma, STAD: Stomach adenocarcinoma, TCL: T-cell lymphoma, TCPL: T-cell prolymphocytic leukemia, TGCT: Testicular Germ Cell Tumors, THCA: Thyroid carcinoma, THYM: Thymoma, TT: Testicular Teratoma, TYST: Testicular Yolk Sac Tumor, UCEC: Uterine Corpus Endometrial Carcinoma, UCS: Uterine Carcinosarcoma, YST: Yolk Sac Tumor

FIGS. 3A, 3B, and 3C show DKK1 overexpression in human PDAC cancer tissues. FIG. 3A shows DKK1 mRNA overexpression in PDAC according to two separate studies (GSE16515 and GSE15471). The data is processed from Human Genome U133 Plus 2.0 Array platform. FIG. 3B shows the DKK1 expression in six human PDAC cell lines (CFPAC1, Mia PaCa-2, PANC-1, PL45, FG, and L3.3) stained with anti-DKK1 mAb (B-7 clone) as assessed by flow cytometry. One lymphoma cancer cell line (Daudi) and PBMC from health donor were served as negative controls. FIG. 3C shows IHC staining with DKK1 mAb in pancreas tissue from ten PDAC patients.

FIG. 4 shows DKK1 gene expression in breast cancer. DKK1 mRNA is significantly overexpressed in triple negative breast cancer (TNBC) than other types (ER, Her2 and PR positive) of breast cancer and normal tissue (TCGA datasets).

FIGS. 5A, 5B, 5C, 5D, 5E, and 5F show DKK1 expression profile in hematological malignancies. DKK1 mRNA is significantly overexpressed in myeloma according to two separate studies GSE13591 (5A) and GSE5900 (5B), follicular lymphoma (GSE12195, 5C), B-cell childhood acute lymphoblastic leukemia from two separate studies GSE13159 (5D) and GSE33315 (5E), pro-B acute lymphoblastic leukemia (5F). Data from TCGA datasets.

FIGS. 6A and 6B DKK1 overexpression in human cancer cell lines and primary cancer cells. FIG. 6A shows DKK1 gene and protein overexpressed in multiple myeloma, lymphoma, breast cancer and prostate cancer cell lines. FIG. 6B shows DKK1 gene and protein overexpressed in primary multiple myeloma and primary B cell lymphoma cells.

FIG. 7 shows high DKK1 expression correlates with cancer progression. PAAD: Pancreatic adenocarcinoma (FIG. 7A), LUAD: Lung adenocarcinoma (FIG. 7B), HNSC: Head and Neck squamous cell carcinoma (FIG. 7C), STAD: Stomach adenocarcinoma (FIG. 7D). Data from TCGA datasets.

FIGS. 8A, 8B, and 8C show the specificity and antibody-binding capacity of DKK1-A2 mAb HMB1 and HMB7. FIG. 8A shows the specificity of HMB1 and HMB7 was detected by ELISA coated with different antigens. FIG. 8B shows the surface binding affinity by DKK1-A2 mAb with different MM cell lines U266 (HLA-A*0201 positive) and ARP-1 (HLA-A*0201 negative). FIG. 8C shows the antibody-binding capacity to pancreatic cancer and liver cancer cell lines with DKK1-A2 mAb.

FIG. 9 shows the affinity of HMB1 and HMB7 measured by a surface plasmon resonance (SPR) sensorgram. Results showed that the affinity (KD) of HMB1 and HMB7 is 1.76×10⁻⁸ and 2.78×10⁻⁶, respectively.

FIG. 10 shows IHC staining with DKK1, HLA-A2 and DKK1-A2 mAb in normal tissue array. We purchased tissue arrays from US Biomax, Inc, which contain 35 tissue types including bone marrow and human organs from 3 individuals on one slide and were stained with DKK1, HLA-A2, or our C2 mAb. IHC staining showed that DKK1 protein was absent from normal tissues except tonsil, testis, ovary, and GI track at a low level, and no DKK1-A2 complex expression was detected in them except tonsil.

FIGS. 11A, 11B, 11C, and 11D show IHC staining with DKK1, HLA-A2 and DKK1-A2 mAb in lung non-small cell carcinoma NSCLC tissue array with normal lung tissue. We purchased NSCLC and normal lung tissue arrays from US Biomax, stained them with anti-human DKK1 mAb, and showed that DKK1 protein is highly expressed by all human NSCLC tumors (n=40) but none of normal lung tissues (n=10) (11A). Importantly, although all NSCLC tumors express DKK1 protein (11B), a portion of NSCLC patients (8/40) or normal lung donors (4/10) are HLA-A2⁺ (11C). DKK1-A2 C2 mAb binds to all 8 (DKK1⁺) HLA-A2⁺ NSCLC tumors but none of 4 (DKK1⁻) HLA-A2⁺ normal lung tissues (11D).

FIGS. 12A. 12B, and 12C show DKK1, HLA-A2 and DKK1(P20)/HLA-A2 expressed in cancer cells. FIG. 12A shows the intracellular DKK1, surface HLA-A2 and DKK1(P20)/HLA-A2 expression in six human NSCLC cell lines stained with anti-DKK1 mAb (B-7 clone), anti-HLA-A2 and anti-DKK1-A2 as assessed by flow cytometry. HLA-A2 positive and negative PBMC were served as control. FIG. 12B shows the intracellular DKK1, surface HLA-A2 and DKK1(P20)/HLA-A2 expression in multiple myeloma cell lines by anti-DKK1 mAb (B-7 clone), anti-HLA-A2 mAb and anti-DKK1-A2 mAb as assessed by flow cytometry. FIG. 12C show IHC staining with DKK1, HLA-A2 and DKK1-A2 mAb in ten PADC tissues.

FIGS. 13A, 13B, 13C, and 13D show apoptosis of different cancer cells induced by DKK1-A2 mAb in vitro. FIG. 13A shows apoptosis of myeloma cell line U266 and lymphoma cell line Jeko-1 induced by DKK1-A2 mAb HMB1, HMB7 and C2. FIG. 13B shows apoptosis of breast cancer cell lines MBA-MB-MD231 and MCF-7 induced by DKK1-A2 mAb HMB1, HMB7 and C2. FIG. 13C shows apoptosis of prostate cancer cell lines LNcap-LN3 and PC-3 (HLA-A2⁺) induced by DKK1-A2 mAb HMB1, HMB7 and C2. FIG. 13D shows apoptosis of pancreatic cancer cell lines CFP-AC1 and PANC1 induced by DKK1-A2 mAb HMB1, HMB7 and C2. PBS and mouse IgG as control. Error bars=SEM. *p<0.05; **p<0.01.

FIGS. 14A and 14B show ADCC and CDC of different cancer cells induced by DKK1-A2 mAb in vitro. FIG. 14A shows Cr-51 release assay detected ADCC of breast cancer cell lines MCF-7 and MBA-MB-MD231, prostate cancer cell lines LNcap-LN3 and PC-3 (HLA-A2⁺), and pancreatic cancer cell lines CFP-AC1 and PANC1 induced by DKK1-A2 mAb HMB1, HMB7 and C2, mouse isotype IgG1 as control. Error bars=SEM.*p<0.05; **p<0.01. Error bars=SEM.*p<0.05; **p<0.01. FIG. 14B shows Cr-51 release assay detected CDC of breast cancer cell lines MCF-7 and MBA-MB-MD231, prostate cancer cell lines LNcap-LN3 and PC-3 (HLA-A2′), and pancreatic cancer cell lines CFP-AC1 and PANC1 induced by DKK1-A2 mAb HMB1, HMB7 and C2, mouse isotype IgG1 as control. Error bars=SEM.*p<0.05; **p<0.01. Error bars=SEM.*p<0.05; **p<0.01.

FIGS. 15A, 15B, and 15C show ADCC and CDC of different cancer cells induced by humanized DKK1-A2 mAb in vitro. FIG. 15A shows surface binding to pancreatic cancer cell lines CFP-AC1, BxPC3 and PANC1 and liver cancer cell line HepG2 with DKK1-A2 mAb C2, and humanized C2 hC2-843 and hC2-844. FIG. 15B shows Cr-51 release assay detected ADCC of breast cancer cell lines MCF-7 and MBA-MB-MD231, prostate cancer cell lines LNcap-LN3 and PC-3 (HLA-A2⁺), and pancreatic cancer cell lines CFP-AC1 and PANC1 induced by grafted C2, and humanized C2 hC2-843 and hC2-844, human isotype IgG1 as control. Error bars=SEM.*p<0.05: **p<0.01. Error bars=SEM.*p<0.05; **p<0.01. FIG. 15C shows Cr-51 release assay detected CDC of breast cancer cell lines MCF-7 and MBA-MB-MD231, prostate cancer cell lines LNcap-LN3 and PC-3 (HLA-A2*), and pancreatic cancer cell lines CFP-AC1 and PANC1 induced by grafted C2, and humanized C2 hC2-843 and hC2-844, human isotype IgG1 as control. Error bars=SEM.*p<0.05; **p<0.01. Error bars=SEM.*p<0.05; **p<0.01.

FIGS. 16A, 16B, and 16C show immunotherapy of HMB1 mAb in breast cancer xenograft mouse model. SCID mice were challenged subcutaneously with MBA-MB-231 breast cancer cells, followed by treatment with intraperitoneal injections (every 3 days for a total of six injections) of 100 mg DKK1-A2 mAb or 100 mg mouse IgG1 (mIgG) or PBS. Tumor burdens were monitored by IVIS. Mice were euthanized when subcutaneous tumors reached 225 mm² or when mice became moribund. Shown are bioluminescence images (16A), tumor burdens (16B) and survival (16C) of mice receiving different treatments. Error bars=SEM.*p<0.05; **p<0.01.

FIGS. 17A, 17B, 17C, 7D, 17E, and 17F show DKK1-A2 CAR-T specific killing DKK1(P20)/HLA-A2 complex positive pancreatic cancer cell. FIG. 17A shows a schematic of DKK1-A2 CAR (C2 clone) construct and CD19 CAR (FMC63 clone). FIG. 17B shows a schematic of the recognition of DKK1(P20)/HLA-A2 complex by DKK1-A2 CAR-T cells. (17C) and (17D) two DKK1/HLA-A2 double positive pancreatic cancer cells can be specifically recognized and killed by DKK1-A2 CAR-T cells. DKK1-A2 CAR-T cells have no cytotoxicity on DKK1/HLA-A2 double negative lymphoma cancer cells Daudi (17E) and HLA-A2 positive PBMCs (17F). Specific cytotoxicity of CAR-T cells was measured by Luciferase reporter assays. HLA-A2 positive PBMC pre-stained with Violet dye, after incubated with effector T cells for 12 hours, cell viability was measure by flow cytometry. E/T is the ratio of effector to target cells. CD19 CAR T cell as a control.

FIGS. 18A, 18B, 18C, 18D, 18E, and 18F shows DKK1-A2 CAR-T cells eliminate established pancreatic cancer (CFPAC-1) in vivo. FIG. 18A shows a diagram of experimental design of PDAC (CFPAC-1) xenograft model. FIG. 18B shows a representative tumor images from mice treated with the indicated T cells or PBS on six weeks after CAR-T infusion. FIG. 18C shows tumor burden measured by bioluminescence at indicated time after CAR-T cell infusion. FIG. 18D shows tumor burden (total flux) quantified by photons/s in mice and (18E) tumor size captured by Caliper were recorded and analyzed at indicated time after CAR-T cell infusion. FIG. 18F shows the bodyweight of mice has no significant change after CAR-T cells infusion. CD19 CAR T cell as a control.

FIGS. 19A, 19B, 19C, 19D, and 19E show DKK1-A2 CAR-T cells eliminate established pancreatic cancer (PL45) in vivo. FIG. 19A shows diagram of experimental design of PDAC (PL45) xenograft model. FIG. 19B shows tumor burden measured by bioluminescence at indicated time (once week) after CAR-T cell infusion. FIG. 19C shows tumor burden (total flux) quantified by photons/s in mice and (19D) tumor size captured by Caliper were recorded and analyzed at indicated days after CAR-T cell infusion. FIG. 19E shows the bodyweight of mice has no significant change after CAR-T cells infusion. CD19 CAR T cell as a control.

FIGS. 20A, 20B, 20C, and 20D show DKK1-A2 CAR-T cells suppress tumor progression in PDAC orthotropic xenograft model. FIG. 20A shows a diagram of experimental design of DKK1-A2 CAR-T anti-tumor activity in CFPAC-1 orthotropic xenograft model. FIG. 20B shows tumor burden measured by bioluminescence at indicated time (once week) after CAR-T cell infusion. FIG. 20C shows tumor burden (total flux) quantified by photons/s in mice and (20D) the bodyweight of mice has no significant change after CAR-T cells infusion.

FIGS. 21A and 21B show DKK1-A2 CAR-T-specific killing DKK1/HLA-A2 positive breast cancer cells. FIG. 21A shows two DKK1/HLA-A2 double positive breast cancer cell lines can be specifically recognized and killed by DKK1-A2 CAR-T cells. DKK1-A2 CAR-T cells have no cytotoxicity on MDA-MD-468 (HLA-A2 negative) and Daudi (HLA-A2 and DKK1 double negative). Specific cytotoxicity of CAR-T cells was measured by Luciferase reporter assays. E/T is the ratio of effector to target cells. FIG. 20B shows IFN γ and TNF α were detected after those cancer cells incubated with indicated CAR-T cells for 12 hours. CD19 CAR T cell as a control.

FIGS. 22A, 22B, 22C, and 22D show breast carcinoma suppressed by DKK1-A2 CAR-T cells in subcutaneous xenograft model. FIG. 22A shows a diagram of experimental design of DKK1-A2 CAR-T anti-tumor activity in MDA-MB-231 subcutaneous xenograft model. FIG. 22B shows tumor burden measured by bioluminescence at indicated time (once week) after CAR-T cell infusion. FIG. 22C shows tumor burden (total flux) quantified by photons/s in mice and (22D) the bodyweight of mice has no significant change after CAR-T cells infusion.

FIGS. 23A, 23B, 23C, 23D, and 23E show DKK1-A2 CAR-T cells eliminate established NSCLC cancer (H441) metastasis model. FIG. 23A shows diagram of experimental design of NSCLC (NCI-H441 cells) model. FIG. 23B shows tumor burden measured by bioluminescence at indicated time (once week) after CAR-T cell infusion. FIG. 23C shows tumor burden (total flux) quantified by photons/s in mice. FIG. 23D shows the bodyweight of mice was recorded after CAR-T cells infusion. FIG. 23E shows Kaplan-Meier survival curves of tumor-bearing mice after treatment CAR-T cells. CD19 CAR T cell as a control.

FIGS. 24A, 24B, 24C, and 24D show humanized C2 and murine C2-derived DKK1-A2 CAR-T cells efficiently suppress orthotopically implanted breast carcinoma. DKK1-A2 CAR-T cells were generated using either murine C2 (C2) or humanized C2 (hC2) sequences. FIG. 24A shows a diagram of experimental design of DKK1-A2 CAR-T anti-tumor activity in MDA-MB-231 orthotropic xenograft model. FIG. 24B shows tumor burden measured by bioluminescence at indicated time (once week) after CAR-T cell infusion. FIG. 24C shows tumor burden (total flux) quantified by photons/s in mice and (24D) the bodyweight of mice has no significant change after CAR-T cells infusion. CD19 CAR T as a control.

FIGS. 25A, 25B, 25C, and 25D show humanized C2 and murine C2-derived DKK1-A2 CAR-T cells efficiently suppress cancer cell growth in established U266 multiple myeloma model. DKK1-A2 CAR-T cells were generated using either murine C2 (C2) or humanized C2 (hC2) sequences. FIG. 25A shows diagram of experimental design of U266 model. FIG. 25B shows tumor burden of human lambda IgG measured by blood ELISA (once week) after CAR-T cell infusion. FIG. 25C shows Kaplan-Meier survival curves of tumor-bearing mice after treatment CAR-T cells in U266 model. FIG. 25D show tumor burden measured by bioluminescence at indicated time (once week) after CAR-T cell infusion.

FIGS. 26A, 26B, 26C, and 26D show humanized C2 and murine C2-derived DKK1-A2 CAR-T cells efficiently suppress cancer cell growth in established KMS-12-BM multiple myeloma model. DKK1-A2 CAR-T cells were generated using either murine C2 (C2) or humanized C2 (hC2) sequences. FIG. 26A shows a diagram of experimental design of KMS-12-BM (HLA-A2 positive) model. FIG. 26B shows tumor burden (total flux) quantified by photons/s in mice. FIG. 26C shows Kaplan-Meier survival curves of tumor-bearing mice after treatment with CAR-T cells. FIG. 26D shows tumor burden measured by bioluminescence at indicated time (once week) after CAR-T cell infusion.

FIGS. 27A, 27B, and 27C show humanized C2 and murine C2-derived DKK1-A2 CAR-T cells efficiently suppress cancer cell growth in established MM.1s multiple myeloma model. DKK1-A2 CAR-T cells were generated using either murine C2 (C2) or humanized C2 (hC2) sequences. FIG. 27A shows a diagram of experimental design of DKK1-A2 CAR-T anti-tumor activity in MM.1s (HLA-A2 positive) xenograft model. FIG. 27B show tumor burden (total flux) quantified by photons/s in mice. FIG. 27C show Kaplan-Meier survival curves of tumor-bearing mice after treatment with CAR-T cells.

FIGS. 28A, 28B, and 28C show the design of T cell dependent bispecific antibodies targeting DKK1-A2 (DKK1-A2-TBD). (28A) BiTE (28B) Cross BiTE (28C) Tand BiTE.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

As used herein, the term “immunotoxin” has its general meaning in the art. By “immunotoxin”, it is meant a chimeric protein made of an antibody or modified antibody or antibody fragment (also called in the present application “antibody”), attached to a fragment of a toxin. The antibody of the immunotoxin is covalently attached to the fragment of a toxin. For use with the disclosed immunotoxins, the toxin ca be diphtheria toxin, Pseudomonas exotoxin, A chain of ricin, ribosome inactivating proteins gelonin, saporin, bouganin, pokeweed antiviral protein, and dodecandron.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular DKK1-A2 binding molecule is disclosed and discussed and a number of modifications that can be made to a number of molecules including the DKK1-A2 binding molecule are discussed, specifically contemplated is each and every combination and permutation of DKK1-A2 binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The Wnt signaling pathway is critical in development and homeostasis of the human body. The Wnt signaling pathways are a group of signal transduction pathways which begin with proteins that pass signals into a cell through cell surface receptors. The Wnt signaling pathways use either nearby cell-cell communication (paracrine) or same-cell communication (autocrine). They are highly evolutionarily conserved in animals, which means they are similar across animal species from fruit flies to humans. The Dickkopf family (Dkks) is a canonical Wnt signaling pathway antagonist, and play varied roles in human physiology. This pathway plays a central role in bone development and formation. Besides modulating the canonical Wnt pathway, Dkks can also interfere with noncanonical Wnt signaling and exert Wnt-independent activities, suggesting context-dependent functions.

Dysfunction of the Dkks family has been implicated in the development and progression of cancer and has been shown to be highly expressed by cancer patients including hematologic (such as myeloma, lymphomas, and leukemia) and solid (such as prostate, lung, breast, and pancreatic cancers) tumors but is absent from normal organs or tissues except placenta and prostate. For example, Dickkopf-1 (DKK1) is broadly expressed in myeloma cells but highly restricted in normal tissues. DKK1 plays a critical role in several aspects of bone biology and actively participates in regulating myeloma bone disease by inhibiting osteoblasts and by activating osteoclasts. Secretion of DKK1 by myeloma cells has been demonstrated to be a major factor in inhibiting osteoblast precursors and thus bone regeneration.

The DKK1 P20 and P66v peptides bind with HLA-A2 molecule-specific cytotoxic T cells and specifically kill myeloma and other cancer cells that express DKK1 and HI-A-A2. However, this is not found in HLA-A2⁺ normal cells, indicating that DKK1+ tumor cells naturally express these peptides in the context of HLA-A2 molecules on their surface. Therefore, DKK1 is a widely expressed, tumor-associated antigen that can be targeted for immunotherapy.

To develop novel immunotherapies for PDAC and NSCLC, we have been exploring whether targeting Dickkpf-1 (DKK1), a secreted protein and tumor-associated antigen (TAA), can be applied to treat PDAC patients. DKK1 mRNA is absent from most human normal tissues except placenta, bladder, esophagus and prostate (NCBI, Human Protein Atlas, and our studies). However, high levels of DKK1 mRNA (TCGA, ONCOMINE) and protein are found in various human cancers including PDAC (>95% tumor cells from all 10 randomly selected, surgically removed human PDAC but none of their adjacent normal pancreatic tissues were stained highly positive for DKK1 protein.

As cancer cells, but not normal cells, express on their surface DKK1 peptides/HLA-A2 molecules in HLA-A2⁺ cancer patients, the DKK1 peptide P20/HLA-A2 was synthesized and used it to immunized mice. Hybridomas were obtained which secrete mAbs that recognize the soluble and cell surface-expressing DKK1 P20/HLA-A2 complex. These mAbs bind specifically with DKK1-expressing, HLA-A2⁺ human cancer cells but not HLA-A2⁺ blood cells or normal tissue cells. Thus, because DKK1 is specifically expressed by human cancer cells but not by normal cells, these mAbs can specifically target cancer cells, but not normal cells in HLA-A2⁺ cancer patients. Most if not all therapeutic mAbs kill their target cells via mediating antibody-dependent, cell-mediated cytotoxicity (ADCC) and/or complement-dependent cytotoxicity (CDC). These mAbs can be used as therapeutic mAbs to treat patients with, such as but not limited to, breast, lung, prostate and pancreatic cancers, and myeloma, lymphomas, and leukemia and kill the cancer cells via mediating antibody-dependent cellular cytotoxicity (ADCC) and complement dependent cytotoxicity (CDC), or by delivering toxin to cancer cells.

In one aspect disclosed herein are antigen binding molecules (such as, for example, RNAi, peptide, protein, chimeric antigen receptor (CAR) T cell, CAR NK cell, CAR macrophage (CARMA), siRNA, immunotoxin, diabody, antibody, or a functional antibody fragment) that selectively binds to Dickkpf-1 (DKK1) peptide P20 in the context of HLA-A2.

Also disclosed herein are antigen binding molecules comprising one or more of the heavy chain complementarity determining regions as set forth in SEQ ID NO: 1, SEQ ID NO: 2, SEQ ID NO. 3, SEQ ID NO: 7, SEQ ID NO: 8 SEQ ID NO: 9, SEQ ID NO: 25. SEQ ID NO: 26, and/or SEQ ID NO: 27 (such as, for example, antigen binding molecules comprising a heavy chain complementarity determining regions (CDRs) 1, 2, and 3 as set forth in SEQ ID NO. 1, SEQ ID NO: 2, SEQ ID NO: 3, respectively, including, but not limited to a heavy chain variable domain as set forth in SEQ ID NO: 14; antigen binding molecules comprising a heavy chain complementarity determining regions (CDRs) 1, 2, and 3 as set forth in SEQ ID NO: 7, SEQ ID NO: 8, SEQ ID NO: 9, respectively, including, but not limited to a heavy chain variable domain as set forth in SEQ ID NO: 18; or antigen binding molecules comprising a heavy chain complementarity determining regions (CDRs) 1, 2, and 3 as set forth in SEQ ID NO: 25, SEQ ID NO: 26, SEQ ID NO: 27, respectively, including, but not limited to a heavy chain variable domain as set forth in SEQ ID NO: 22) and/or one or more of the light chain complementarity determining regions as set forth in SEQ ID NO: 4, SEQ ID NO: 5, SEQ ID NO: 6, SEQ ID NO: 10, SEQ ID NO: 11, SEQ ID NO: 12, SEQ ID NO: 28, SEQ ID NO: 29, and/or SEQ ID NO: 30 (such as, for example, antigen binding molecules comprising a light chain complementarity determining regions (CDRs) 1, 2, and 3 as set forth in SEQ ID NO: 4, SEQ ID NO: 5, and SEQ ID NO: 6, respectively, including, but not limited to antigen binding molecules comprising a light chain variable domain as set forth in SEQ ID NO: 16; antigen binding molecules comprising a light chain complementarity determining regions (CDRs) 1, 2, and 3 as set forth in SEQ ID NO: 10, SEQ ID NO: 11, and SEQ ID NO: 12, respectively, including, but not limited to antigen binding molecules comprising a light chain variable domain as set forth in SEQ ID NO: 20; or antigen binding molecules comprising a light chain complementarity determining regions (CDRs) 1, 2, and 3 as set forth in SEQ ID NO: 428, SEQ ID NO: 29, and SEQ ID NO: 30, respectively, including, but not limited to antigen binding molecules comprising a light chain variable domain as set forth in SEQ ID NO: 24). For example, the antigen binding molecule can comprise SEQ ID NO: 14 and SEQ ID NO: 16; SEQ ID NO: 18 and SEQ ID NO: 20; or a humanized variable heavy chain and humanized variable light chain as set forth in SEQ ID NO: 22 and SEQ ID NO: 24. In some aspects, the antigen binding molecule can be conjugated to a detectable moiety such as a label (such as, for example, a fluorescent dye, a member of a binding pair, a metal, or an epitope tag).

In one aspect disclosed herein are antigen binding molecules wherein antigen binding molecule comprises a CAR T cell that comprises a Th9 polarized T cell.

Also disclosed herein are antigen binding molecules, wherein antigen binding molecule comprises a bi-specific antibody (such as, for example, BiTE, Cross BiTE, or Tand BiTE (see FIG. 28) wherein the first antigen binding molecules bind DKK1 peptide P20 in the context of MHC HLA-A2 and the second antigen binding molecule binds CD3.

### 1. Antibodies

### (1) Antibodies Generally

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (I), based on the amino acid sequences of their constant domains.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with DKK1 P20 in the context of HLA-A2 (i.e., a DKK1 P20/HLA-A2 complex also referred to herein as DKK1-A2). The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “antibodies” is used herein in a broad sense and includes both polyclonal 5 and monoclonal antibodies. As used herein, the term “antibody” encompasses, but is not limited to, whole immunoglobulin (i.e., an intact antibody) of any class. In addition to intact immunoglobulin molecules, also included in the term “antibodies” are fragments or polymers of those immunoglobulin molecules, and human or humanized versions of immunoglobulin molecules or fragments thereof. It should be understood that the “antibody” can be monoclonal antibodies, polyclonal antibodies, chimeric antibodies, bi-specific antibodies (diabody, BiTE Cross BiTE, or Tand BiTE), or tri-specific antibody (triabody).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. In one aspect, disclosed herein are hybridoma cell lines expressing any of the antigen binding molecules disclosed herein (for example, antigen binding molecules that specifically bind to DKK1 peptide P20 in the context MHC HLA-A2 also referred to herein as a DKK1-A2 complex binding molecule).

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain.

Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, sFv, VHH, and the like, including hybrid fragments. Thus, fragments of the antibodies that retain the ability to bind their specific antigens are provided. For example, fragments of antibodies which maintain DKK1 P20/HLA-A2 complex binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies. A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

Disclosed are hybridoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The anti-DKK1 P20/HLA-A2 complex antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Homology/Identity

It is understood that one way to define any known variants and derivatives or those that might arise, of the disclosed genes and proteins herein is through defining the variants and derivatives in terms of homology to specific known sequences. For example SEQ ID NO: 13, SEQ ID NO: 15, SEQ ID NO: 17; SEQ ID NO: 19, SEQ ID NO: 21 and SEQ ID NO: 23 sets forth a particular sequence of variable heavy or variable light chains that specifically bind DKK1 P20 peptide in the context of MHC HLA-A2 and SEQ ID NO: 14, SEQ ID NO: 16, SEQ ID NO: 18; SEQ ID NO: 20, SEQ ID NO: 22 and SEQ ID NO: 24 sets forth a particular sequence of the protein encoded by SEQ ID NO: 13, SEQ ID NO: 15, SEQ ID NO: 17: SEQ ID NO: 19, SEQ ID NO: 22 and SEQ ID NO: 24, respectively. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Peptides

### a) Protein Variants

As discussed herein there are numerous variants of the SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 14, SEQ ID NO. 16, SEQ ID NO: 18; SEQ ID NO: 20, SEQ ID NO: 22, SEQ ID NO: 24, SEQ ID NO: 25, SEQ ID NO: 26, SEQ ID NO: 27, SEQ ID NO: 28, SEQ ID NO: 29, and/or SEQ ID NO: 30 that are known and herein contemplated. In addition, there are derivatives of the SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO. 11, SEQ ID NO: 12, SEQ ID NO: 14, SEQ ID NO: 16, SEQ ID NO: 18; SEQ ID NO: 20, SEQ ID NO: 22, SEQ ID NO: 24, SEQ ID NO: 25, SEQ ID NO: 26, SEQ ID NO: 27, SEQ ID NO: 28, SEQ ID NO: 29, and/or SEQ ID NO: 30 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90°/% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review): Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 4. Method of Treating Cancer

It is understood and herein contemplated that the disclosed DKK1-A2 complex binding molecules can be used to treat any disease where uncontrolled cellular proliferation occurs such as cancers. Thus, in one aspect, disclosed herein are methods of treating, inhibiting, reducing, decreasing, ameliorating, and or preventing a cancer and/or metastasis (such as, for example, pancreatic cancer, non-small cell lung carcinoma, breast cancer, myeloma, or leukemia) in a subject comprising administering to the subject any of the antigen binding molecules disclosed herein. For example, disclosed herein are methods of treating, inhibiting, reducing, decreasing, ameliorating, and or preventing a cancer and/or metastasis (such as, for example, pancreatic cancer, non-small cell lung carcinoma, breast cancer, myeloma, or leukemia) in a subject comprising administering to the subject a therapeutically effective amount of an antibody, bi-specific antibody (including, but not limited to a BiTE, Cross BiTE, and/or Tand BiTE), immunotoxin, or CAR T cell that specifically binds to DKK1 peptide P20 in the context of MHC HLA-A2.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, drenocortical carcinoma, bladder urothelial carcinoma, breast invasive carcinoma, cervical squamous cell carcinoma, endocervical adenocarcinoma, cholangiocarcinoma, CML: Chronic Myelomonocyte Leukemia, colon adenocarcinoma, lymphoid neoplasm diffuse large B-cell lymphoma, esophageal carcinoma, fibrous histiocytoma, follicular lymphoma, glioblastoma multiforme, kidney renal clear cell carcinoma, kidney renal papillary cell carcinoma, brain lower grade glioma, Liver hepatocellular carcinoma, large cell lung carcinoma, lung adenocarcinoma, lung squamous cell carcinoma, mantle cell lymphoma, myxofibrosarcoma, mixed germ cell tumor, monoclonal gammopathy of undertermined significance, multiple myeloma, ovarian serous cystadenocarcinoma, pro-B acute lymphoblastic leukemia, pheochromocytoma and paraganglioma, rectum adenocarcinoma, sarcoma, skin cutaneous melanoma, stomach adenocarcinoma, T-cell prolymphocytic leukemia, testicular germ cell tumors, thyroid carcinoma, thymoma, testicular teratoma, testicular yolk sac tumor, uterine corpus endometrial carcinoma, uterine carcinosarcoma, yolk sac tumor or pancreatic cancer

In one aspect, it is understood and herein contemplated that successful treatment of a cancer in a subject is important and doing so may include the administration of additional treatments. Thus, the disclosed methods of detecting/diagnosing and methods of treating, reducing, inhibiting, decreasing, ameliorating and/or preventing a cancer and/or metastasis can include or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Additionally, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### 5. Immunoassays and Fluorochromes

In addition to directing an antibody, immunotoxin, or CAR T cell to a therapeutic target, it is understood and herein contemplated that the disclosed DKK1-A2 complex binding molecules can be used to detect the presence of a cancer in a subject. In one aspect, disclosed herein are methods of detecting a cancer in a subject comprising a) obtaining a tissue sample of a suspected cancerous tissue from the subject; and b) assaying (such as, for example, by enzyme linked immunosorbent assays (ELISAs), enzyme linked immunospot assay (ELISpot). radioimmunoassays (RIA), radioimmune precipitation assays (RIPA), immunobead capture assays, and/or flow cytometry) for the presence or overexpression of DKK1 with any of the antigen binding molecules of any of claims 1-6; wherein the presence of the DKK1 in the tissue sample not present in a negative control tissue sample or overexpression of the DKK1 in the tissue sample relative to a negative control tissue sample indicates the presence of a cancer in the subject. It is understood and herein contemplated that the binding molecules used for detection of a cancer can be conjugated to a detectable moiety such as a label (such as, for example, a fluorescent dye, a member of a binding pair, a metal, or an epitope tag).

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR, Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue, Calcium Crimson -; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO, DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange, Y66F; Y66H; Y66W; Yellow GFP, YFP; YO-PRO-1; YO-PRO 3, YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are known to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immune complexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immune complex (antigen/antibody) formation. Detection of the immune complex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immune complex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immune complexes between the test sample and the originally bound material, and subsequent washing, the occurrence of even minute amounts of immune complexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immune complex with a labeled antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional, this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialized chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include color coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodesrm particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, CA; Biosite, San Diego, CA). In addition to the conventional antibodies, Fab and scFv fragments, single V-domains from camelids (VHH) or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, CA).

Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### 6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

We generated T-cell receptor (TCR)-like murine and humanized mAbs recognizing DKK1 (P20) peptide in the context of human HLA-A2 (DKK1-A2 complex; patent: UTSC.P1418US.P1), because HLA-A2 is the most frequent serologic type in all ethnic groups and expressed in 47.6% of human populations (49.6% Caucasians, 34.6% African-American, 46.9% Hispanic, and 36.0% Asian/Pacific Islanders). These mAbs bind with high affinity to all HLA-A2⁺ human PDAC cell lines and to >95% tumor cells of examined HLA-A2⁺ PDAC patients, but not to (HLA-A2⁺) normal (35 different type) human tissues except tonsil, indicating that high levels of DKK1-A2 complexes exist on almost all HLA-A2⁺ PDAC tumor cells but not normal cells and thus are an excellent and safe target for immunotherapy. Using the mAb sequences, we generated human DKK1-A2 CAR-T cells that effectively and specifically lyse DKK1⁺HLA-A2⁺ PDAC cell lines but not normal cells in vitro. More importantly, DKK1-A2 CAR-T cells, compared with control (CD19-targeted) CAR-T cells, are highly therapeutic against established human PDAC and NSCLC in NSG mice.

We also discovered that adoptive T-cell therapy can be significantly improved to treat solid tumors by polarizing T cells under Th9 condition. Th9-polarized murine CD4 or CD8 T cells secrete mainly IL-9 and exert greater antitumor efficacy in vivo compared to the classical Th1/Th17 or Tc1/CTL cells, due to their long-persistence, highly proliferative, and better tumor-infiltrating/survival capacities. Recently, we further showed that human CD19 or GPC3 CAR-T cells polarized under the Th9 condition had enhanced antitumor activities against solid human tumors in vivo compared to classically polarized CAR-T cells.

TABLE 3
CDRs of heavy chain and light chain variable
sequences of antibody C2, HMB1 and HMB7

| Chain | CDR | C2, hC2-843 | HMB1 | HMB7 |
|---|---|---|---|---|
| Heavy | CDR1 | GFTFSSFG | DFAVT | DTYMH |
| | | (SEQ ID | (SEQ ID | SEQ ID |
| | | NO: 25) | NO: 1) | NO: 7) |
| | CDR2 | ISSGSSTI | VIWGGGS | RIDPANG |
| | | (SEQ ID | TYYNSA | DTKYDP |
| | | NO: 26) | LKS | KFQG |
| | | | SEQ ID | SEQ ID |
| | | | NO: 2) | NO: 8) |
| | CDR3 | ARPYYY | TTPIYFD | GRYYYG |
| | | GSTYDA | YDHALD | TSSFDY |
| | | MDY | Y SEQ ID | SEQ ID |
| | | (SEQ ID | NO: 3) | NO: 9) |
| | | NO: 27) | | |
| Light | CDR1 | QDVGST | RSSQSLT | KAWQDV |
| | | (SEQ ID | NSYGNT | DTAVA |
| | | NO: 28) | YLS | SEQ ID |
| | | | SEQ ID | NO: 10) |
| | | | NO: 4) | |
| | CDR2 | WTS | GISNRFS | WASTRHT |
| | | (SEQ ID | SEQ ID | SEQ ID |
| | | NO: 29) | NO: 5) | NO: 11) |
| | CDR3 | QKFGSY | LQGTH | QQYSS |
| | | PLT | QPYT | YPYT |
| | | (SEQ ID | SEQ ID | SEQ ID |
| | | NO: 30) | NO: 6) | NO: 12) |

### D. SEQUENCES

Antibody sequences of HMB1 were listed as below:

SEQ ID NO: 13:
Heavy chain: DNA sequence (423 bp)
ATGGCTGTCCTGGGGCTGCTTCTCTGCCTGGTGACGTTCCCAAGC
TGTGTCCTGTCCCAGGTACAGCTGAAGGAGTCAGGACCTGGCCTG
GTGGCGCCCTCACAGAGCCTGTCCATCACATGCACTGTCTCAGGG
TTCTCATTAACCGACTTTGCTGTGACCTGGATTCGCCAGCCTCCA
GGAAAGGGTCTGGAGTGGCTGGGAGTAATCTGGGGTGGTGGAAGC
ACATACTATAATTCAGCTCTCAAATCCAGACTGAGCATCAGCAAG
GACAACTCCAAGAGCCAAGTTTTCTTAAAAATGAACAGTCTTCAA
ACTGATGACACAGCCATGTACTACTGTGCCAAAACAACCCCTATC
TATTTTGATTACGACCATGCTTTGGACTACTGGGGTCAAGGAACC
TCAGTCACCGTCTCCTCA
SEQ ID NO: 14:
Heavy chain: Amino acid sequence (141 aa)
MAVLGLLLCLVTFPSCVLSQVQLKESGPGLVAPSQSLSITCTVSG
FSLTDFAVTWIRQPPGKGLEWLGVIWGGGSTYYNSALKSRLSISK
DNSKSQVFLKMNSLQTDDTAMYYCAKTTPIYFDYDHALDYWGQGT
SVTVSS
SEQ ID NO: 15:
Light chain: DNA sequence (396 bp)
ATGAATTTGCCTGTTCATCTCTTGGTGCTTCTGTTGTTCTGGATT
CCTGCTTCCAGAGGTGATGTTGTGGTGACTCAAACTCCACTCTCC
CTGCCTGTCAGCTTTGGAGATCAAGTTTCTATCTCTTGCAGGTCT
AGTCAGAGTCTTACAAACAGTTATGGGAACACCTATTTGTCTTGG
TACCTGCACAAGCCTGGCCAGTCTCCACAGCTCCTCATCTATGGG
ATTTCCAACAGATTTTCTGGGGTGCCAGACAGGTTCAGTGGCAGT
GGTTCAGGGACAGATTTCACACTCAAGATCAGCACAATAAAGCCT
GAGGACTTGGGAATGTATTACTGCTTACAAGGTACACATCAGCCG
TACACGTTCGGAGGGGGGACCAAGCTGGAAATAAAA
SEQ ID NO: 16:
Light chain: Amino acid sequence (132 aa)
MNLPVHLLVLLLFWIPASRGDVVVTQTPLSLPVSFGDQVSISCRS
SQSLTNSYGNTYLSWYLHKPGQSPQLLIYGISNRFSGVPDRFSGS
GSGTDFTLKISTIKPEDLGMYYCLQGTHQPYTFGGGTKLEIK

Antibody sequences of HMB7 were listed as below:

SEQ ID NO: 17:
Heavy chain: DNA sequence (420 bp)
ATGAAATGCAGCTGGGTTATCTTCTTCCTGATGGCAGTGGTTACA
GGGGTCAATTCAGAGGTTCAGCTGCAGCAGTCTGGGGCAGAGCTT
GTGAAGCCAGGGGCCTCAGTCAAGTTGTCCTGCACAGCTTCTGGC
TTCAACATTAAAGACACCTATATGCACTGGGTGAAGCAGAGGCCT
GAACAGGGCCTGGAGTGGATTGGAAGGATTGATCCTGCGAATGGT
GATACTAAATATGACCCGAAGTTCCAGGGCAAGGCCACTATAACA
GCAGACACATCCTCCAACACAGCCTACCTGCACCTCAGCAGCCTG
ACATCTGAGGACACTGCCGTCTATTACTGTGCTAGAGGGCGTTAT
TACTACGGTACTAGCTCCTTTGACTACTGGGGCCAAGGCACCACT
CTCACAGTCTCCTCA
SEQ ID NO: 18:
Heavy chain: Amino acid sequence (140 aa)
MKCSWVIFFLMAVVTGVNSEVQLQQSGAELVKPGASVKLSCTASG
FNIKDTYMHWVKQRPEQGLEWIGRIDPANGDTKYDPKFQGKATIT
ADTSSNTAYLHLSSLTSEDTAVYYCARGRYYYGTSSFDYWGQGTT
LTVSS
SEQ ID NO: 19:
Light chain: DNA sequence (381 bp)
ATGGAGACACATTCTCAGGTCTTTGTATACATGTTGCTGTGGTTG
TCTGGTGTTGAAGGAGACATTGTGATGACCCAGTCTCACAAATTC
ATGTCCACATCAGTAGGAGACTGGGTCAGCATCACCTGCAAGGCC
TGGCAGGATGTGGATACTGCTGTAGCCTGGTATCAACAGAAACCA
GGGCAATCTCCTAAACTACTGATTTACTGGGCATCCACCCGGCAC
ACTGGAGTCCCTGATCGCTTCACAGGCAGTGGATCTGGGACAGAT
TTCACTCTCACCATTAGCAATGTGCAGTCTGAAGACTTGGCAGAT
TATTTCTGTCAGCAATATAGCAGCTATCCGTACACGTTCGGAGGG
GGGACCAAGCTGGAAATAAAA
SEQ ID NO: 20:
Light chain: Amino acid sequence (127 aa)
METHSQVFVYMLLWLSGVEGDIVMTQSHKFMSTSVGDWVSITCKA
WQDVDTAVAWYQQKPGQSPKLLIYWASTRHTGVPDRFTGSGSGTD
FTLTISNVQSEDLADYFCQQYSSYPYTFGGGTKLEIK

Antibody sequences of humanized C2 (hC2-843) were listed as below:

SEQ ID NO: 21:
Heavy chain: DNA sequence (1416 bp)
ATGGGCTGGAGCTGGATCCTGCTGTTCCTCCTGAGCGTGACAGCA
GGAGTGCACAGCGAGGTGCAGCTGGTGGAGTCTGGAGGAGGACTG
GTGCAGCCAGGAGGCTCTCTGCGGCTGAGCTGCGCCGCCTCCGGC
TTCACCTTCAGCTCCTTCGGCATGCACTGGGTGAGGCAGGCACCT
GGCAAGGGACTGGAGTGGGTGGCCTATATCTCTAGCGGCTCCTCT
ACCATCTACTATGCCGACACAGTGAAGGGCCGGTTTACCATCTCT
AGAGATAACGCCAAGAATAGCCTGTACCTGCAGATGAACTCCCTG
AGGGCCGAGGACACAGCCGTGTACTATTGTGCCCGCCCCTACTAT
TACGGCTCCACCTATGACGCCATGGATTACTGGGGCCAGGGCACC
CTGGTGACAGTGAGCTCCGCTAGCACCAAGGGCCCATCGGTCTTC
CCCCTGGCACCCTCCTCCAAGAGCACCTCTGGGGGCACAGCGGCC
CTGGGCTGCCTGGTCAAGGACTACTTCCCCGAACCGGTGACGGTG
TCGTGGAACTCAGGCGCCCTGACCAGCGGCGTGCACACCTTCCCG
GCTGTCCTACAGTCCTCAGGACTCTACTCCCTCAGCAGCGTGGTG
ACCGTGCCCTCCAGCAGCTTGGGCACCCAGACCTACATCTGCAAC
GTGAATCACAAGCCCAGCAACACCAAGGTGGACAAGAAAGTTGAG
CCCAAATCTTGTGACAAAACTCACACATGCCCACCGTGCCCAGCA
CCTGAACTCCTGGGGGGACCGTCAGTCTTCCTCTTCCCCCCAAAA
CCCAAGGACACCCTCATGATCTCCCGGACCCCTGAGGTCACATGC
GTGGTGGTGGACGTGAGCCACGAAGACCCTGAGGTCAAGTTCAAC
TGGTACGTGGACGGCGTGGAGGTGCATAATGCCAAGACAAAGCCG
CGGGAGGAGCAGTACAACAGCACGTACCGTGTGGTCAGCGTCCTC
ACCGTCCTGCACCAGGACTGGCTGAATGGCAAGGAGTACAAGTGC
AAGGTCTCCAACAAAGCCCTCCCAGCCCCCATCGAGAAAACCATC
TCCAAAGCCAAAGGGCAGCCCCGAGAACCACAGGTGTACACCCTG
CCCCCATCCCGGGATGAGCTGACCAAGAACCAGGTCAGCCTGACC
TGCCTGGTCAAAGGCTTCTATCCCAGCGACATCGCCGTGGAGTGG
GAGAGCAATGGGCAGCCGGAGAACAACTACAAGACCACGCCTCCC
GTGCTGGACTCCGACGGCTCCTTCTTCCTCTACAGCAAGCTCACC
GTGGACAAGAGCAGGTGGCAGCAGGGGAACGTCTTCTCATGCTCC
GTGATGCATGAGGCTCTGCACAACCACTACACGCAGAAGAGCCTC
TCCCTGTCTCCGGGTAAATGA
SEQ ID NO: 22:
Heavy chain: Amino acid sequence (471 aa)
MGWSCIILFLVATATGVHSEVQLVESGGGLVQPGGSLRLSCAASG
FTFSSFGMHWVRQAPGKGLEWVAYISSGSSTIYYADTVKGRFTIS
RDNAKNSLYLQMNSLRAEDTAVYYCARPYYYGSTYDAMDYWGQGT
LVTVSSASTKGPSVFPLAPSSKSTSGGTAALGCLVKDYFPEPVTV
SWNSGALTSGVHTFPAVLQSSGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIA VE
WESNGQPENNYKTTPPVLDSDGSFFLYSKLTVDKSRWQQGNVFSC
SVMHEALHNHYTQKSLSLSPGK
SEQ ID NO: 23:
Light chain: DNA sequence (702 bp)
ATGGGCTGGAGCTGGATCCTGCTGTTCCTCCTGAGCGTGACAGCA
GGAGTGCACAGCGACATCCAGCTGACCCAGTCTCCTAGCTTTCTG
TCCGCCTCTGTGGGCGACAGGGTGACCATCACATGCAAGGCCAGC
CAGGATGTGGGCTCCACAGTGGCCTGGTACCAGCAGAAGCCCGGC
AAGGCCCCTAAGCTGCTGATCTATTGGACCTCTACAAGGCACACC
GGAGTGCCAAGCCGGTTCAGCGGATCCGGATCTGGAACAGAGTTC
ACCCTGACAATCAGCTCCCTGCAGCCAGAGGATTTCGCCACCTAC
TTTTGTCAGAAGTTCGGATCCTATCCACTGACCTTCGGCGGAGGC
ACAAAGCTGGAGATCAAGCGAACGGTGGCTGCACCATCTGTCTTC
ATCTTCCCGCCATCTGATGAGCAGTTGAAATCTGGAACTGCCTCT
GTTGTGTGCCTGCTGAATAACTTCTATCCCAGAGAGGCCAAAGTA
CAGTGGAAGGTGGATAACGCCCTCCAATCGGGTAACTCCCAGGAG
AGTGTCACAGAGCAGGACAGCAAGGACAGCACCTACAGCCTCAGC
AGCACCCTGACGCTGAGCAAAGCAGACTACGAGAAACACAAAGTC
TACGCCTGCGAAGTCACCCATCAGGGCCTGAGCTCGCCCGTCACA
AAGAGCTTCAACAGGGGAGAGTGTTAG
SEQ ID NO: 24:
Light chain: Amino acid sequence (233 aa)
MGWSCIILFLVATATGVHSDIQLTQSPSFLSASVGDRVTITCKAS
QDVGSTVAWYQQKPGKAPKLLIYWTSTRHTGVPSRFSGSGSGTEF
TLTISSLQPEDFATYFCQKFGSYPLTFGGGTKLEIKRTVAAPSVF
IFPPSDEQLKSGTASVVCLLNNFYPREAKVQWKVDNALQSGNSQE
SVTEQDSKDSTYSLSSTLTLSKADYEKHKVYACEVTHQGLSSPVT
KSFNRGEC

1. An antigen binding molecule that selectively binds to Dickkpf-1 (DKK1) peptide P20 in the context of HLA-A2.

2. The antigen binding molecule of claim 1, comprising one or more of the heavy chain complementarity determining regions as set forth in SEQ ID NO: 1, SEQ ID NO: 2, SEQ ID NO: 3, SEQ ID NO: 7, SEQ ID NO: 8, SEQ ID NO: 9, SEQ ID NO: 25, SEQ ID NO: 26, and/or SEQ ID NO: 27.

3. The antigen binding molecule of claim 1, comprising a heavy chain variable domain as set forth in SEQ ID NO: 14, SEQ ID NO: 18, or SEQ ID NO: 22.

4. The antigen binding molecule of claim 1, comprising one or more of the light chain complementarity determining regions as set forth in SEQ ID NO: 4, SEQ ID NO: 5, SEQ ID NO: 6, SEQ ID NO: 10, SEQ ID NO: 11, SEQ ID NO:
12, SEQ ID NO: 28, SEQ ID NO: 29, or SEQ ID NO: 30.

5. The antigen binding molecule of claim 1, comprising a light chain variable domain as set forth in SEQ ID NO: 16, SEQ ID NO: 20, or SEQ ID NO: 24.

6. The antigen binding molecule of claim 1, wherein the antigen binding molecule comprises an RNAi, peptide, protein, chimeric antigen receptor (CAR) T cell, CAR NK cell, CAR macrophage (CARMA), siRNA, immunotoxin, diabody, antibody, or a functional antibody fragment.

7. The antigen binding molecule of claim 6, wherein the antigen binding molecule comprises a CAR T cell that comprises a Th9 polarized T cell.

8. The antigen binding molecule of claim 6, wherein h antigen binding molecule comprises a humanized antibody comprising SEQ ID NO: 22 and SEQ ID NO: 24.

9. The antigen binding molecule of claim 6, wherein h antigen binding molecule comprises a bi-specific antibody that binds DKK1 P20 in the context of MHC HLA-A2 and binds CD3.

10. A hybridoma cell line expressing the antigen binding molecule of claim 1.

11. A method of treating a cancer in a subject comprising administering to the subject the antigen binding molecule of claim 1.

12. The method of claim 11, wherein the cancer comprises pancreatic cancer, non-small cell lung carcinoma, breast cancer, myeloma, or leukemia.

13. A method of detecting a cancer in a subject comprising
a. obtaining a tissue sample of a suspected cancerous tissue from the subject; and
b. assaying for the presence or overexpression of DKK1 with the antigen binding molecule of claim 1;
wherein the presence of the DKK1 in the tissue sample not present in a negative control tissue sample or overexpression of the DKK1 in the tissue sample relative to a negative control tissue sample indicates the presence of a cancer in the subject.

14. The method of claim 13, wherein the presence of or amount of antigen binding molecule is determined by enzyme linked immunosorbent assays (ELISAs), enzyme linked immunospot assay (ELISpot). radioimmunoassays (RIA), radioimmune precipitation assays (RIPA), immunobead capture assays, and/or flow cytometry.


SEQUENCE LISTING (30 sequences)

SEQ ID NO: 1 - protein, 5 aa, Artificial Sequence
  synthetic construct
DFAVT

SEQ ID NO: 2 - protein, 16 aa, Artificial Sequence
  synthetic construct
VIWGGGSTYYNSALKS

SEQ ID NO: 3 - protein, 14 aa, Artificial Sequence
  synthetic construct
TTPIYFDYDHALDY

SEQ ID NO: 4 - protein, 16 aa, Artificial Sequence
  synthetic construct
RSSQSLTNSYGNTYLS

SEQ ID NO: 5 - protein, 7 aa, Artificial Sequence
  synthetic construct
GISNRFS

SEQ ID NO: 6 - protein, 9 aa, Artificial Sequence
  synthetic construct
LQGTHQPYT

SEQ ID NO: 7 - protein, 5 aa, Artificial Sequence
  synthetic construct
DTYMH

SEQ ID NO: 8 - protein, 17 aa, Artificial Sequence
  synthetic construct
RIDPANGDTKYDPKFQG

SEQ ID NO: 9 - protein, 12 aa, Artificial Sequence
  synthetic construct
GRYYYGTSSFDY

SEQ ID NO: 10 - protein, 11 aa, Artificial Sequence
  synthetic construct
KAWQDVDTAVA

SEQ ID NO: 11 - protein, 7 aa, Artificial Sequence
  synthetic construct
WASTRHT

SEQ ID NO: 12 - protein, 9 aa, Artificial Sequence
  synthetic construct
QQYSSYPYT

SEQ ID NO: 13 - protein, 423 aa, Artificial Sequence
  synthetic construct
ATGGCTGTCCTGGGGCTGCTTCTCTGCCTGGTGACGTTCCCAAGCTGTGTCCTGTCCCAGGTACAGCTGAAGGAGTCAGGACCTGGCCTGGTGGCGCCCTCACAGAGCCTGTCCATCACATGCACTGTCTCAGGGTTCTCATTAACCGACTTTGCTGTGACCTGGATTCGCCAGCCTCCAGGAAAGGGTCTGGAGTGGCTGGGAGTAATCTGGGGTGGTGGAAGCACATACTATAATTCAGCTCTCAAATCCAGACTGAGCATCAGCAAGGACAACTCCAAGAGCCAAGTTTTCTTAAAAATGAACAGTCTTCAAACTGATGACACAGCCATGTACTACTGTGCCAAAACAACCCCTATCTATTTTGATTACGACCATGCTTTGGACTACTGGGGTCAAGGAACCTCAGTCACCGTCTCCTCA

SEQ ID NO: 14 - protein, 141 aa, Artificial Sequence
  synthetic construct
MAVLGLLLCLVTFPSCVLSQVQLKESGPGLVAPSQSLSITCTVSGFSLTDFAVTWIRQPPGKGLEWLGVIWGGGSTYYNSALKSRLSISKDNSKSQVFLKMNSLQTDDTAMYYCAKTTPIYFDYDHALDYWGQGTSVTVSS

SEQ ID NO: 15 - protein, 396 aa, Artificial Sequence
  synthetic construct
ATGAATTTGCCTGTTCATCTCTTGGTGCTTCTGTTGTTCTGGATTCCTGCTTCCAGAGGTGATGTTGTGGTGACTCAAACTCCACTCTCCCTGCCTGTCAGCTTTGGAGATCAAGTTTCTATCTCTTGCAGGTCTAGTCAGAGTCTTACAAACAGTTATGGGAACACCTATTTGTCTTGGTACCTGCACAAGCCTGGCCAGTCTCCACAGCTCCTCATCTATGGGATTTCCAACAGATTTTCTGGGGTGCCAGACAGGTTCAGTGGCAGTGGTTCAGGGACAGATTTCACACTCAAGATCAGCACAATAAAGCCTGAGGACTTGGGAATGTATTACTGCTTACAAGGTACACATCAGCCGTACACGTTCGGAGGGGGGACCAAGCTGGAAATAAAA

SEQ ID NO: 16 - protein, 132 aa, Artificial Sequence
  synthetic construct
MNLPVHLLVLLLFWIPASRGDVVVTQTPLSLPVSFGDQVSISCRSSQSLTNSYGNTYLSWYLHKPGQSPQLLIYGISNRFSGVPDRFSGSGSGTDFTLKISTIKPEDLGMYYCLQGTHQPYTFGGGTKLEIK

SEQ ID NO: 17 - protein, 420 aa, Artificial Sequence
  synthetic construct
ATGAAATGCAGCTGGGTTATCTTCTTCCTGATGGCAGTGGTTACAGGGGTCAATTCAGAGGTTCAGCTGCAGCAGTCTGGGGCAGAGCTTGTGAAGCCAGGGGCCTCAGTCAAGTTGTCCTGCACAGCTTCTGGCTTCAACATTAAAGACACCTATATGCACTGGGTGAAGCAGAGGCCTGAACAGGGCCTGGAGTGGATTGGAAGGATTGATCCTGCGAATGGTGATACTAAATATGACCCGAAGTTCCAGGGCAAGGCCACTATAACAGCAGACACATCCTCCAACACAGCCTACCTGCACCTCAGCAGCCTGACATCTGAGGACACTGCCGTCTATTACTGTGCTAGAGGGCGTTATTACTACGGTACTAGCTCCTTTGACTACTGGGGCCAAGGCACCACTCTCACAGTCTCCTCA

SEQ ID NO: 18 - protein, 140 aa, Artificial Sequence
  synthetic construct
MKCSWVIFFLMAVVTGVNSEVQLQQSGAELVKPGASVKLSCTASGFNIKDTYMHWVKQRPEQGLEWIGRIDPANGDTKYDPKFQGKATITADTSSNTAYLHLSSLTSEDTAVYYCARGRYYYGTSSFDYWGQGTTLTVSS

SEQ ID NO: 19 - protein, 381 aa, Artificial Sequence
  synthetic construct
ATGGAGACACATTCTCAGGTCTTTGTATACATGTTGCTGTGGTTGTCTGGTGTTGAAGGAGACATTGTGATGACCCAGTCTCACAAATTCATGTCCACATCAGTAGGAGACTGGGTCAGCATCACCTGCAAGGCCTGGCAGGATGTGGATACTGCTGTAGCCTGGTATCAACAGAAACCAGGGCAATCTCCTAAACTACTGATTTACTGGGCATCCACCCGGCACACTGGAGTCCCTGATCGCTTCACAGGCAGTGGATCTGGGACAGATTTCACTCTCACCATTAGCAATGTGCAGTCTGAAGACTTGGCAGATTATTTCTGTCAGCAATATAGCAGCTATCCGTACACGTTCGGAGGGGGGACCAAGCTGGAAATAAAA

SEQ ID NO: 20 - protein, 127 aa, Artificial Sequence
  synthetic construct
METHSQVFVYMLLWLSGVEGDIVMTQSHKFMSTSVGDWVSITCKAWQDVDTAVAWYQQKPGQSPKLLIYWASTRHTGVPDRFTGSGSGTDFTLTISNVQSEDLADYFCQQYSSYPYTFGGGTKLEIK

SEQ ID NO: 21 - protein, 1416 aa, Artificial Sequence
  synthetic construct
ATGGGCTGGAGCTGGATCCTGCTGTTCCTCCTGAGCGTGACAGCAGGAGTGCACAGCGAGGTGCAGCTGGTGGAGTCTGGAGGAGGACTGGTGCAGCCAGGAGGCTCTCTGCGGCTGAGCTGCGCCGCCTCCGGCTTCACCTTCAGCTCCTTCGGCATGCACTGGGTGAGGCAGGCACCTGGCAAGGGACTGGAGTGGGTGGCCTATATCTCTAGCGGCTCCTCTACCATCTACTATGCCGACACAGTGAAGGGCCGGTTTACCATCTCTAGAGATAACGCCAAGAATAGCCTGTACCTGCAGATGAACTCCCTGAGGGCCGAGGACACAGCCGTGTACTATTGTGCCCGCCCCTACTATTACGGCTCCACCTATGACGCCATGGATTACTGGGGCCAGGGCACCCTGGTGACAGTGAGCTCCGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAAGTTGAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTAAATGA

SEQ ID NO: 22 - protein, 471 aa, Artificial Sequence
  synthetic construct
MGWSCIILFLVATATGVHSEVQLVESGGGLVQPGGSLRLSCAASGFTFSSFGMHWVRQAPGKGLEWVAYISSGSSTIYYADTVKGRFTISRDNAKNSLYLQMNSLRAEDTAVYYCARPYYYGSTYDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 702 aa, Artificial Sequence
  synthetic construct
ATGGGCTGGAGCTGGATCCTGCTGTTCCTCCTGAGCGTGACAGCAGGAGTGCACAGCGACATCCAGCTGACCCAGTCTCCTAGCTTTCTGTCCGCCTCTGTGGGCGACAGGGTGACCATCACATGCAAGGCCAGCCAGGATGTGGGCTCCACAGTGGCCTGGTACCAGCAGAAGCCCGGCAAGGCCCCTAAGCTGCTGATCTATTGGACCTCTACAAGGCACACCGGAGTGCCAAGCCGGTTCAGCGGATCCGGATCTGGAACAGAGTTCACCCTGACAATCAGCTCCCTGCAGCCAGAGGATTTCGCCACCTACTTTTGTCAGAAGTTCGGATCCTATCCACTGACCTTCGGCGGAGGCACAAAGCTGGAGATCAAGCGAACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGTTAG

SEQ ID NO: 24 - protein, 233 aa, Artificial Sequence
  synthetic construct
MGWSCIILFLVATATGVHSDIQLTQSPSFLSASVGDRVTITCKASQDVGSTVAWYQQKPGKAPKLLIYWTSTRHTGVPSRFSGSGSGTEFTLTISSLQPEDFATYFCQKFGSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 8 aa, Artificial Sequence
  synthetic construct
GFTFSSFG

SEQ ID NO: 26 - protein, 8 aa, Artificial Sequence
  synthetic construct
ISSGSSTI

SEQ ID NO: 27 - protein, 15 aa, Artificial Sequence
  synthetic construct
ARPYYYGSTYDAMDY

SEQ ID NO: 28 - protein, 6 aa, Artificial Sequence
  synthetic construct
QDVGST

SEQ ID NO: 29 - protein, 3 aa, Artificial Sequence
  synthetic construct
WTS

SEQ ID NO: 30 - protein, 9 aa, Artificial Sequence
  synthetic construct
QKFGSYPLT